(19) United States
(12) Patent Application Publication
(10) Pub. No.: US 2003/0086940 A1
(43) Pub. Date: May 8, 2003

(54) Engineered recombinant molecule that regulates humoral and cellular effector functions of the immune system

(76) Inventors: Cristina Costa, New Haven, CT (US); Maryellen Pizzolato, Old Saybrook, CT (US); William Fodor, Madison, CT (US)

Correspondence Address: Stephen A. Saxe, Alexion Pharmaceuticals, Inc., 352 Knotter Drive, Cheshire, CT, 06410, US

(21) Appl. No.: 10/225,519

(22) Filed: Aug. 20, 2002

Related U.S. Application Data
(63) Continuation-in-part of application No. 09/928,267, filed on Aug. 10, 2001.

(51) Int. Cl. A61K 39/00; C12Q 1/68; C07H 21/04; C12P 21/02; C12N 5/06; C07K 14/435; C12N 9/99

(52) U.S. Cl. 424/185.1; 435/69.1; 435/320.1; 435/325; 435/6; 530/395; 536/23.1; 435/184

(57) ABSTRACT

Recombinant chimeric molecules that include at least a domain capable of regulating the humoral effector functions of the immune system and another domain capable of regulating the cellular effector functions of the immune system are provided. Recombinant DNA constructs having DNA sequences encoding the above mentioned chimeric proteins are provided. Cloning vectors incorporating the above DNA constructs and cells transformed with the vectors and host cells containing such vectors are also provided. Transgenic cells, tissues, organs and animals incorporating the above-mentioned chimeric molecules are provided.

### CROSS-REFERENCE TO RELATED APPLICATIONS

This application is a continuation-in-part of U.S. application Ser. No. 09/928,267 filed Aug. 10, 2001 which claims priority from International Application No. PCT/US00/29151 filed Oct. 21, 2000, and is related to U.S. Application Serial No. 60/161,186 filed Oct. 22, 1999, all of which are incorporated herein by reference.

### STATEMENT CONCERNING FEDERALLY SPONSORED RESEARCH

This application was made with Government support from National Institutes of Standards and Technology—Advanced Technology Program under Grant No. 70NANB8H4054. The federal government may have certain rights in this invention.

### TECHNICAL FIELD

Chimeric proteins capable of conferring resistance to humoral and cellular mechanisms of immune attack and more particularly chimeric proteins having at least a domain derived from a complement inhibitor protein and a domain derived from a T-cell inhibitor protein or an NK cell inhibitor protein are provided. DNA constructs encoding such chimeric proteins and methods of preparing such chimeric proteins are disclosed. Methods of using such chimeric proteins, including in the prevention or treatment of rejection of xenotransplants are described.

### BACKGROUND OF THE INVENTION

Research in pig-to-primate xenotransplantation aims to solve the increasing shortage of organs for human allotransplantation and to develop new cell- and tissue-based therapies. The possibility to obtain large quantities of pig cells, tissues and organs under controlled conditions, in opposition to the shortage of human organs for allotransplantation, has generated interest in the development of new xenogeneic therapies (Auchincloss and Sachs, 1998; Lu et al., 1994; Lambrigts et al., 1998; Edge et al., 1998). However, several immune barriers need to be addressed in order to achieve clinical success (Auchincloss and Sachs, 1998; Lu et al., 1994; Lambrigts et al., 1998; Edge et al., 1998). Both humoral and cellular defense mechanisms mediate the rejection of transplanted cells, tissues, and organs during xenotransplantation. The survival of organs and tissues during xenotransplantation requires multiple immunosuppressive strategies to inhibit antibody reactivity, complement activation, and cellular rejection. These immune mechanisms can lead to hyperacute rejection, delayed xenograft rejection and acute cellular rejection. These phases of rejection are characterized by the time of onset, the immunobiologic pathways involved and different pathologic characteristics.

Hyperacute rejection (HAR) can occur within minutes post transplantation and is initiated by recipient antibody deposition on the donor endothelium. Antibody deposition leads to the activation of host complement and the coagulation cascade, resulting in thrombosis, ischemia, and necrosis (Auchincloss, 1988; Platt and Bach, 1991; Somerville and D'Apice, 1993; Dalmasso et al., 1992; Lu et al., 1994; Auchincloss and Sachs, 1998). Hyperacute rejection has been addressed in pig-to-primate transplant models using various treatment regimes including: systemic antibody removal (Leventhal et al., 1995; Leventhal et al., 1993a), systemic complement inhibition (Pruitt et al., 1994), combination therapy with immunosuppressive drugs (Leventhal et al., 1994; Davis et al., 1996) and transgenic donor pig organs that express human complement inhibitors (McCurry et al., 1995; Diamond et al., 1996) or human H-transferase (Koike et al., 1996; Sandrin et al., 1995). The application of engineered organs has proven efficacious in controlling this first wave of immune attack.

A major hurdle that needs to be addressed to achieve engraftment of xenogeneic cells, tissues and organs is delayed xenograft rejection (DXR) (Goodman et al., 1997; Platt et al., 1998; Lin et al., 1998; Candinas et al., 1996). The etiology of DXR is complex and needs further elucidation. Delayed xenograft rejection (DXR) can be a form of accelerated vascular rejection and involves deposition of IgG and IgM classes of antibody on the donor endothelium, leading to Type II endothelial cell activation (Leventhal et al., 1993b; Blakely et al., 1994; Candinas et al., 1996). Antibody deposition can also lead to antibody dependent cell-mediated cytotoxicity (ADCC) mechanisms where recipient monocytes, macrophages and NK cells can induce endothelial cell activation, the generation of a procoagulant environment, intravascular thrombosis and fibrin deposition (Watier et al., 1996; Kumagai-Braesch et al., 1998). The role of antibodies in DXR was clearly demonstrated by Lin et al. (1998), whereby removal of immunoglobulin resulted in increased survival of the heterotopically transplanted CD59/DAF double transgenic hearts. Histologic analysis of the transgenic tissue showed reduced thromboses, reduced endothelial cell damage and reduced fibrin deposition.

Acute cellular rejection (ACR) can occur within days following transplantation and is predominantly a T cell-mediated response to donor antigens. ACR is characterized by lymphocytic and macrophage cellular infiltration and endothelialitis (Auchincloss and Sachs, 1998; Millan et al., 1997). T cells of the CD4⁺ lineage have been identified as mediators of the primary cellular xenograft response in the direct and indirect antigen presentation pathways. In addition, acute cell mediated rejection by CD4⁺ T cells can occur in the absence of NK, B, CD8+ T cells (Koulmanda et al., 1995; Pierson et al., 1989; Simeonovic et al., 1990; Wecker et al., 1994). T cell activation during xenotransplantation has been studied extensively and several groups have addressed cell mediated immune reactivity with immunosuppressive drug therapy (Kawauchi et al., 1993; Matsumiya et al., 1996; Zhong et al., 1996) with T cell specific antibody reagents (Chavin et al., 1992; Pierson et al., 1989; Inverardi et al., 1992) and with recombinant immunosuppressive protein ligands, such as CD152Ig (Tran et al., 1997; Chahine et al., 1995; Pearson et al., 1994; Walunas et al., 1994; Sun et al., 1997; Lenschow et al., 1992; Larsen et al., 1996; Steurer et al., 1995; Blazar et al., 1995).

Many porcine cell types and all vascularized organs transplanted into primate models are rejected by both humoral and cellular-mediated mechanisms (Dalmasso et al., 1992; Kobayashi et al., 1997). Major advances have been made in abrogating the humoral component that leads to hyperacute rejection, mainly through complement inhibition (Fodor et al., 1994; Kroshus et al., 1996; Cozzi et al., 1997; Byrne et al., 1997). However, even in the presence of complement inhibitors and systemic immunosuppression, xenografts are rejected due to delayed xenograft rejection (DXR) (Lambrigts et al., 1998; Kobayashi et al., 1997; Goodman et al., 1997; Platt et al., 1998; Lin et al., 1998; Candinas et al., 1996). DXR is characterized by a strong humoral response, type II endothelial activation and an acute cellular infiltration comprising NK cells and macrophages (Kobayashi et al., 1997; Goodman et al., 1997; Platt et al., 1998; Lin et al., 1998; Candinas et al., 1996). The importance of NK cell-mediated xenograft rejection has been demonstrated in small animal models (Candinas et al., 1996; Lin et al., 1997; Xia et al., 2000), while its contribution to the rejection process in the pig-to-primate model is less defined (Kobayashi et al., 1997; Itescu et al., 1998).

It is known in the art that T cells, also called T lymphocytes, are a part of the vertebrate immune system. T cells recognize foreign pathogens (such as bacteria, viruses, or parasites), tissues, and/or organs, and help the immune system process them (causing what is referred to in the art as a cellular immune response), generally clearing the pathogens from the body. T cell activation is not only dependent on antigen recognition, but also on engagement of costimulatory molecules found on antigen presenting cells (APCs). The costimulatory signal that determines whether antigen recognition leads to full T cell activation or to T cell unresponsiveness, i.e., anergy, is that generated by the interaction of CD28 on the T cells with B7 on the APCs; see for example Harding et al. (1992) who demonstrated in vitro that cross-linking of the CD28 molecule can rescue T cells from becoming anergic. It is further known that both B7-1 (CD80) and B7-2 (CD86) molecules on APCs provide critical costimulatory signals in T cell activation through their binding with the CD28 molecule on the T cell and, moreover, that antigens presented in the absence of such costimulatory signals result in T cell anergy.

It is also known in the art that the complement system (known in the art to be part of the humoral immune system) is an interaction of at least 25 plasma proteins and membrane cofactors which act in a multistep, multiprotein cascade sequence in conjunction with other immunological systems of the body to defend against intrusion of foreign cells and viruses. Complement components achieve their immune defensive functions by interacting in a series of intricate but precise enzymatic cleavage and membrane binding events. The resulting complement cascade leads to the production of products with opsonic, immunoregulatory and lytic functions.

CD59 is known to be the archetypical inhibitor of part of the complement system known as the C5b-9 membrane attack complex (MAC). When activated and in the absence of inhibition, the C5b-9 MAC can cause potentially deleterious cell activation and cell lysis. CD59 is a human glycoprotein, the nucleotide and amino acid sequences for which are set forth in SEQ ID NOs:9 and 10. CD59 is found associated with the membranes of cells including human erythrocytes, lymphocytes, and vascular endothelial cells. It serves to prevent assembly of functional MACs and thus protects cells from complement-mediated activation and/or lysis and is tethered to the outer surface of the cell membrane by a glycosyl-phosphatidylinositol (GPI) anchor. See, for example, Sims et al., U.S. Pat. No. 5,135,916.

Chimeric proteins, also referred to in the art as fusion proteins, are hybrid proteins which combine at least parts of two or more precursor proteins or peptides. Chimeric proteins may be produced by recombinant technology, i.e., by fusing at least a part of the coding sequence of one gene to at least a part of the coding sequence of another gene. The fused gene may then be used to transform a suitable organism which then expresses the fusion protein.

Bi-functional complement inhibitors including fusion proteins constructed from the C3 family of inhibitor proteins (such as DAF (decay accelerating factor) or CD55; CR1 or CD35; MCP or CD46; or factor H (see, e.g., Liszewski et al., 1996; Liszewski and Atkinson, 1998; Morgan and Meri, 1994; Fearon, 1979; Seya et al., 1986; Nicholson-Weller et al., 1982; Pangburn and Müller-Eberhard, 1983)) and the C5b-9 family of inhibitor proteins (such as CD59) are known. See U.S. Pat. Nos. 5,847,082; 5,624,837; and 5,627,264. It has been demonstrated that the CD59 moiety in a DAF-CD59 chimeric molecule functions to inhibit MAC when expressed membrane proximal and anchored through its endogenous GPI linkage. See Fodor et al. (1995). The Fodor et al. (1995) publication teaches that chimeric complement inhibitors retained conformational epitopes and functional activity.

Various techniques have been investigated to regulate T-cell interactions and immune responses mediated by such interactions. It is known that CTLA4 (which is also referred to as CD152, the terms CTLA4 and CD152 being used interchangeably throughout this disclosure including in the Figures) is a T-cell surface receptor that associates with the B7-1 (CD80) and B7-2 (CD86) molecules which are expressed on antigen-presenting cells. See for example Hathcock et al. (1994). It is further known that this association establishes the molecular basis for an important T-cell co-stimulatory pathway, the primary function of which is to induce T-cell cytokine production and proliferation following exposure to antigen. See for example Linsley et al. (1991). U.S. Pat. No. 5,434,131 identifies the CTLA4 receptor as a ligand for the B7 antigen and discloses methods for using soluble fusion proteins to regulate immune responses, including T-cell interactions. U.S. Pat. No. 5,773,253 provides CTLA 4 mutant molecules as ligands for the B7 antigen and methods for expressing the mutant molecules as soluble functional molecules which regulate T-cell interactions. U.S. Pat. Nos. 5,844,095 and 5,851,795 describe methods of expressing CTLA4 as an immunoglobulin fusion protein, methods of preparing hybrid CTLA4 fusion proteins, and methods of using the soluble fusion proteins, fragments and derivatives thereof, to regulate cellular immune responses and T-cell interactions. U.S. Pat. No. 5,869,050 discloses methods of blocking T-cell activation using anti-B7 monoclonal antibodies to overcome allograft transplant rejection and/or graft versus host disease, as well as to prevent or treat rheumatoid arthritis.

However, no single molecule exists today which can be used in the prevention or treatment of both humoral and cellular rejection of xenotransplants. No such molecules exist that when expressed provide the cell with both protection from human serum complement and inhibit T-cell and NK cell activation. Such a molecule would be particularly advantageous in the production of transgenic animals. Microinjection of recombinant DNA into the pronuclei of animal ova for generating transgenic animals is known. However, since this technology is dependent on random integration of DNA, it is a complex procedure to achieve targeted cellular expression of two distinct heterologous proteins by the simultaneous microinjection of their respective DNAs (such as would be required if CTLA4 inhibitory activity and CD59 inhibitory activity were to be achieved through the use of individual entities).

Moreover, as described above, currently the soluble form of CTLA4 or CTLA4Ig fusion proteins are used to regulate cellular immune responses and T-cell interactions by binding B7-1 and B7-2 in trans when administered exogenously. Therefore, it would be of additional advantage if the CTLA4 moiety could bind endogenously expressed B7-1 and B7-2 molecules in cis and block the co-stimulation necessary for engagement of human CD28 expressed on T-cells, thereby protecting the xenotransplanted porcine cell from the human cellular immune response by rendering the human T-cells unresponsive to the porcine target cell.

Similarly, it will be of further advantage if NK cell activation could be blocked thereby limiting delayed xenograft rejection. Additionally, it will be an advantage if complement-mediated lysis is inhibited.

### SUMMARY OF THE INVENTION

Recombinant chimeric molecules that include at least a domain capable of regulating the humoral effector functions of the immune system and another domain capable of regulating the cellular effector functions of the immune system now surprisingly have been engineered. Suitable domains capable of regulating the humoral effector functions of the immune system include complement inhibitory domains, such as a C5b-9 and/or C3 inhibitory domain. Suitable domains capable of regulating the cellular effector functions of the immune system include T-cell inhibitory domains and NK cell inhibitory domains. In one embodiment, a membrane bound chimeric molecule that includes functional domains derived from CTLA4 (human or porcine) and human CD59 is provided. In another embodiment, a membrane bound chimeric molecule that includes functional domains derived from CTLA4 (human or porcine) and human DAF is provided. The chimeric molecules are shown to exhibit regulatory activity in cis.

Recombinant DNA constructs having DNA sequences encoding the above mentioned chimeric proteins are provided. Cloning vectors incorporating the above DNA constructs and cells transformed with the vectors and host cells containing such vectors are also provided. Transgenic cells, tissues, organs, and animals incorporating the above-mentioned chimeric molecules are provided.

Methods for preparing a DNA construct including a DNA sequence encoding a domain of CD59 containing the complement regulatory domain operably linked to a DNA sequence encoding a T-cell or NK cell inhibitory domain are provided. Also provided are methods of manufacturing the above described chimeric proteins by transforming a cell with a suitable cloning vector including a DNA construct encoding the chimeric protein, and expressing the gene such that the resulting protein is expressed on the cell membrane.

### BRIEF DESCRIPTION OF THE DRAWINGS

FIGS. 1A-C depict diagrammatic representations of recombinant chimeric molecules.

FIGS. 2A-B are fluorescence activated cell sorting (FACS) profiles that demonstrate the expression of the CTLA4 and CD59 domains of the chimeric molecules on the cell surface of transduced PAECs. FIG. 2A represents cells transduced with pBABEpuro. FIG. 2B represents cells transduced with hCTLA4hCD59BABEpuro. The x-axes (FL1-H) are the fluorescence intensity. ctr is the control.

FIGS. 3A-F depict FACS analyses that show the phosphatidyl inositol phospholipase C (PI-PLC) mediated removal of CTLA4 and CD59 domains from transduced PAECs. FIGS. 3A, 3C and 3E represent cells not treated with phosphatidyl inositol phospholipase C (PI-PLC) and FIGS. 3B, 3D and 3F represent cells that were treated with PI-PLC. FIGS. 3A-B represent cells transduced with pBABEpuro vector. FIGS. 3C-D represent cells transduced with hCTLA4hCD59BABEpuro. FIGS. 3E-F represent cells transduced with pCTLA4hCD59BABEpuro. The x-axes (FL1-H) are the fluorescence intensity. ctr is the control.

FIGS. 4A-F show cell surface expression of chimeric molecules. Assays are performed with anti-human or anti-pig CD152 (antiCTLA4) or anti-human CD59 antibodies. The assays are: transduced puromycin control PAECs (FIG. 4A), hCTLA4-hCD59 PAECs (FIG. 4B), transfected puromycin control Balb/c 3T3 cells (FIG. 4C), hCTLA4-hCD59-29 Balb (FIG. 4D), pCTLA4(AS)₃-hCD59-22 Balb (FIG. 4E) and pCTLA4(G)₆-hCD59-54 Balb (FIG. 4F). The x-axes (FL1-H) are the fluorescence intensity.

FIGS. 5A-B show the results of complement mediated dye release assays. Human serum complement incubated with PAECs (FIG. 5A) or Balb/c 3T3 cells (FIG. 5B) expressing either the human or porcine chimeric constructions. FIG. 5A shows untreated vector control PAECs or the hCTLA4-hCD59 transduced PAECs compared to PI-PLC treated PAECs. FIG. 5B shows vector control Balb/c 3T3 as compared to hCTLA4CD59-29, pCTLA4CD59-22 and pCTLA4CD59-54. Percent cell lysis is plotted against the percent of human serum used as the complement source. Each point represents the mean calculated from three experiments.

FIGS. 6A-D show anti CD86/CD80 antibody reactivity. Cis binding of engineered CTLA4 chimeric molecules to surface expressed CD86/CD80 molecules is removed by enzymatic removal of the chimeric molecule thereby revealing CD86/CD80 (FIG. 6A) or by competitive binding of anti CTLA4 to the chimeric molecule (FIG. 8B). In FIG. 6A, porcine CD86 availability is shown on untreated vector control or hCTLA4CD59 PAEC and compared with PI-PLC treated cells. Murine CD80 availability is shown on vector control Balb/c 3T3 (FIG. 6B), pCTLA4(G)₆-hCD59-22 (FIG. 6C) and pCTLA4(G)₆-hCD59-54 (FIG. 6D) treated with anti-pCTLA4 polyclonal antiserum.

FIG. 7 illustrates results from a Jurkat-PAEC costimulation assay. The reduction in pCD86/hCD28 specific IL-2 secretion from Jurkat cells due to the hCTLA4-CD59 chimeric molecule expressed on PAECs is compared to vector control PAECs in the absence or presence of the reagents indicated. PI-PLC treated PAECs are compared to untreated PAECs. The concentration of IL-2 secreted is plotted on the ordinate. The cell type is indicated on the abscissa.

FIG. 8 shows results form a Jurkat-Balb/c 3T3 costimulation assay. The reduction in mCD80/hCD28 specific IL-2 secretion from Jurkat cells due to either the hCTLA4-CD59 or the pCTLA4-hCD59 chimeric molecules is compared to vector Balb/c 3T3 cells in the absence or presence of the reagents indicated.

FIGS. 9A-H show flow cytometric analyses of PAEC, NK, NK92 and YTS cells. Specific staining towards the indicated molecules is represented with a thick line. The dotted line corresponds to the secondary antibody reactivity (directly conjugated isotype matched antibodies were used as negative controls). The x-axes show the log of the fluorescence intensity and the y-axes indicate relative cell number.

FIG. 10 shows lysis of PAEC mediated by IL-2 activated NK, NK92 and YTS cells. The results are representative of three separate experiments.

FIGS. 11A-F represent NK cell-mediated lysis of control and HT-transgenic PAEC expressing various levels of hCTLA4-CD59. FIGS. 11A, 11C and 11E are for NK92 cells as effector cells. FIGS. 11B, 11D and 11F show the results using IL-2 activated NK cells as effector cells. FIGS. 11A-B are results using PAEC from non-transgenic control pig #51 unmodified, expressing medium hCTLA4-CD59 (hCCm) or high levels of hCTLA4-CD59 (hCCh). FIGS. 11C-D show results using PAEC from HT-transgenic pig #48 transduced with pBABE vector alone (v) or expressing low levels of hCTLA4-CD59 (hCCl). FIGS. 11E-F show results from HT-transgenic pig #49 unmodified or expressing medium levels of hCTLA4-CD59 (hCCm) or high levels of hCTLA4-CD59 (hCCh). In each case the results are representative of three independent experiments.

FIGS. 12A-B show the effect of CD86 blockade on NK92-mediated lysis of control and HT-transgenic PAEC. FIG. 12A shows results using control pig #51 PAECs and FIG. 12B shows results using PAEC from HT-transgenic pig #49. The PAECs were either unmodified or expressed high levels of hCTLA4-CD59 (hCCh). These were preincubated with 5B9.88 anti-CD86 blocking antibody (5B9) or isotype antibody control (IgG) and subsequently challenged with NK92. Results are representative of three independent experiments.

FIGS. 13A-D show flow cytometric analyses of control and HT-transgenic porcine fibroblasts. FIG. 13A shows detection of the H epitope detected with UEAI. FIG. 13B shows the Galα1,3-Gal epitope detected with GS-IB4. FIG. 13C shows CD86 expression as assessed with 4F9.86 antibody. FIG. 13D shows SLA class I expression as assessed by PT85A antibody. All values are expressed as the mean±SE of the mean fluorescence intensity (n=4). *p≦0.05, **p≦0.005.

FIGS. 14A-B show NK cell-mediated lysis of control and HT-transgenic porcine fibroblasts. FIG. 14A shows the results using NK92 cells as effector cells. FIG. 14B shows results using IL-2 activated NK cells as effector cells. The cells are fibroblasts isolated from a control, the HTAT21 and HTAT20 founders, and from an HTAT21F₁ pig. Results are representative of three independent experiments. Similar results were obtained in another four experiments including cells isolated from different animals.

FIGS. 15A-C show the effect of CD86 blockade on NK cell-mediated lysis of control and HTAT20-transgenic fibroblasts. Control and HTAT20-transgenic fibroblasts (FIG. 15A) or the HTAT20 transgenic fibroblasts transduced with vector alone (v) or hCTLA4-CD59 (hCC) (FIG. 15B) were pre-incubated with 5B9.88 anti-CD86 blocking antibody (5B9) or isotype antibody control (IgG). Target cells were subsequently challenged with NK92 cells. FIG. 15C show an evaluation of IL-2 activated NK cell-mediated cytotoxicity towards the control, HTAT20, HTAT20v and HTAT20hCC cells. Results are representative of three independent experiments.

### BRIEF DESCRIPTION OF THE SEQUENCE LISTING

SEQ ID NO:1 is a nucleotide sequence of a porcine CTLA4-human CD59 chimeric molecule including a human CD59 leader sequence and a (G)₆ linker.

SEQ ID NO:2 is an amino acid sequence of a porcine CTLA4-human CD59 chimeric molecule including a human CD59 leader sequence and a (G)₆ linker.

SEQ ID NO:3 is a nucleotide sequence of a human CTLA4-human CD59 chimeric molecule with a human CD59 leader sequence and an (AS)₃ linker.

SEQ ID NO:4 is an amino acid sequence of a human CTLA4-human CD59 chimeric molecule with a human CD59 leader sequence and an (AS)₃ linker.

SEQ ID NO:5 is a nucleotide sequence of porcine CTLA4.

SEQ ID NO:6 is an amino acid sequence of porcine CTLA4.

SEQ ID NO:7 is a nucleotide sequence of human CTLA4.

SEQ ID NO:8 is an amino acid sequence of human CTLA4.

SEQ ID NO:9 is a nucleotide acid sequence for human CD59 (hCD59).

SEQ ID NO:10 is an amino acid sequence for human CD59.

SEQ ID NO:11 is an amino acid sequence for a portion of human DAF.

SEQ ID NO:12 is a peptide linker sequence.

SEQ ID NO:13 is a peptide linker sequence.

SEQ ID NO:14 is a forward oligonucleotide primer used for PCR of human CTLA4.

SEQ ID NO:15 is a reverse oligonucleotide primer used for PCR of human CTLA4.

SEQ ID NO:16 is a degenerate forward oligonucleotide primer used for PCR of porcine CTLA4.

SEQ ID NO:17 is a degenerate reverse oligonucleotide primer used for PCR of porcine CTLA4.

SEQ ID NO:18 is a forward oligonucleotide primer used for PCR of exon 2 of porcine CTLA4.

SEQ ID NO:19 is a reverse oligonucleotide primer used for PCR of exon 2 of porcine CTLA4.

SEQ ID NO:20 is a forward oligonucleotide primer used for PCR of exons 1 and 2 of porcine CTLA4.

SEQ ID NO:21 is a reverse oligonucleotide primer used for PCR of exons 1 and 2 of porcine CTLA4.

SEQ ID NO:22 is a forward oligonucleotide primer used for PCR of exon 2 of porcine CTLA4.

SEQ ID NO:23 is a reverse oligonucleotide primer used for PCR of exon 2 of porcine CTLA4.

SEQ ID NO:24 is a nucleotide sequence of a porcine CTLA4-human CD59 chimeric molecule with a porcine CTLA4 leader sequence and an (AS)₃ linker.

SEQ ID NO:25 is an amino acid sequence of a porcine CTLA4-human CD59 chimeric molecule with a porcine CTLA4 leader and an (AS)₃ linker.

SEQ ID NO:26 is a nucleotide sequence of a porcine CTLA4-human CD59 chimeric molecule with a porcine CTLA4 leader sequence and a (G)₆ linker.

SEQ ID NO:27 is an amino acid sequence of a porcine CTLA4-human CD59 chimeric molecule with a porcine CTLA4 leader sequence and a (G)₆ linker.

### DETAILED DESCRIPTION OF THE INVENTION

It has been found that functional domains capable of regulating the humoral effector functions of the immune system including complement inhibitory domains, such as a C5b-9 inhibitory domain or C3 inhibitory domain, and functional domains capable of regulating the cellular effector functions of the immune system, including T-cell and NK cell inhibitory domains, can advantageously be combined to form a chimeric protein. The chimeric protein can be expressed on a porcine cell surface and can aid in the protection of the porcine cell, after xenotransplantation into a human, from both the human cellular immune response and human complement.

Applying genetic modifications to donor cells, tissues and organs in order to modulate the immune system has unique advantages over systemic immunosuppressive regimens. Engineered xenogeneic cells or allogeneic stem cells that resist immune attack could lead to universal sources of donor tissue that require minimal immunosuppressive drug therapy. Blocking the immune response at multiple steps with a single molecule has additional advantages in that fewer genetic modifications are necessary to achieve enhanced survival.

It is herein demonstrated that hCTLA4-hCD59 or pCTLA4-hCD59 molecules can be expressed on transduced porcine aortic endothelial cells and/or transfected Balb/3T3 cells. The cells expressing the chimeric molecules are resistant to complement-mediated lysis and do not costimulate T cells. By enzymatically digesting the molecule from the cell surface or using antibody blocking experiments, the activity and costimulation inhibition is shown to be specific to the chimeric molecule. Mechanistically, the functional domain, which regulates cellular effector function, binds with molecules expressed on antigen presenting cells. As a consequence of these interactions, these cells elicit significantly less IL-2 production from human T cells as compared to vector control transfected cells. Thus a membrane bound chimeric protein in accordance with the present disclosure (e.g., an hCTLA4-hCD59 or pCTLA4-hCD59 molecule) expressed on pig or mouse cells can inhibit humoral and cellular defense mechanisms.

The phrase “C5b-9 inhibitory activity” is used herein to describe the effects of C5b-9 inhibitor molecules on the complement system and thus includes activities that lead to inhibition of the cell activating and/or lytic function of the membrane attack complex (MAC).

Suitable domains which exhibit C5b-9 inhibitory activity can include the entire amino acid sequence for a naturally occurring C5b-9 inhibitor protein or a portion thereof. For example, the C5b-9 inhibitory sequence can be the mature CD59 molecule (i.e., amino acids 1 through 103 of SEQ ID NO:10). Alternatively, the C5b-9 inhibitory sequence can be a portion of a naturally occurring C5b-9 inhibitor protein, such as CD59. Active portions suitable for use herein can be identified using a variety of assays for C5b-9 inhibitory activity known in the art. See for example Rollins and Sims, 1990; Rollins et al., 1991; Zhao et al., 1991; and Rother et al., 1994. In general, the portion used should have at least about 25% and preferably at least about 50% of the activity of the parent molecule.

Suitable C3 inhibitory domains include the entire amino acid sequence for a naturally occurring C3 inhibitor or a portion thereof, such as one or more SCRs (short consensus repeats) of the C3 inhibitory domain. For example, the C3 inhibitor sequence can be the mature DAF molecule, factor H, membrane cofactor protein or complement receptor 1. Alternatively, the C3 inhibitory domain can be a portion of a naturally occurring C3 inhibitor protein. Following the procedures used to identify functional domains of DAF (Adams et al., 1991), functional domains of other C3 inhibitors can be identified and used herein. In general, the portion used should have at least about 25% and preferably at least about 50% of the activity of the parent C3 inhibitory molecule. Particularly useful portions of mature C3 inhibitor proteins include one or more of the mature molecule's SCRs. These SCRs are normally approximately 60 amino acids in length and have four conserved cysteine residues which form disulfide bonds, as well as conserved tryptophan, glycine, and phenylalanine/tyrosine residues. In one embodiment the C3 inhibitory domain includes SCRs 2 through 4 of DAF (i.e., amino acids 97 through 286 if the leader sequence is included in the numbering or amino acids 63 through 252 shown by the numbering in SEQ ID NO:11).

Suitable domains which exhibit T-cell inhibitory activity can include either at least a portion of the amino acid sequence for naturally occurring porcine CTLA4 (pCTLA4) or at least a portion of the entire amino acid sequence for naturally occurring human CTLA4 (hCTLA4). For example, the amino acid sequence which exhibits T-cell inhibitory activity can be amino acids 38 to 162 of the porcine CTLA4 sequence if including the leader sequence in the numbering, which is amino acids 1-125 as shown by the numbering in SEQ ID NO:6 (amino acids 28-152 of SEQ ID NO:2) or amino acids 38 to 161 of the human CTLA4 sequence (amino acids 1-124 as shown in SEQ ID NO:8 or amino acids 28-151 of SEQ ID NO:4). In general, the portion used should have at least about 25% and preferably at least about 50% of the activity of the parent molecule (Thompson et al., 1989; Shapiro et al., 1998).

The use of the present chimeric molecules in NK cell-mediated cytolysis assays blocks CD86 without triggering CD16-mediated ADCC. The present combination of two inhibitory strategies probably leads to a better appreciation of their effects in the complex NK cell triggering system.

A marked reduction in Galα-1,3-Gal antigen expression on porcine cells provided by the present chimeric molecules together with CD86 blockade led to complete protection from human NK-cell mediated lysis. Complete inhibition of activation signals in the NK cell cytolytic machinery can thus be achieved by the present chimeric molecules.

To test the hypothesis that CD80 and/or CD86 on porcine cells triggers human NK cell activation and killing, a chimeric molecule in accordance with this disclosure (hCTLA4-hCD59) was expressed on the porcine cells to block CD80/CD86-mediated costimulation in cis was utilized. PAEC and porcine fibroblasts constitutively express CD86 in the cell surface and do not express CD80 in resting conditions. Increasing levels of hCTLA4-hCD59 expression in these cells correlated with a reduction in both CD86 accessibility and susceptibility to lysis mediated by human NK cells in the absence of human serum. The use of a specific anti-CD86 blocking antibody and the NK cell lines NK92 and YTS further confirmed the involvement of CD86, but not CD80, in triggering NK cell-mediated cytotoxicity of porcine cells. To investigate the combinatorial role of CD86 and the carbohydrate epitope Galα1,3-Gal on NK cell activation, a chimeric molecule in accordance with the disclosure (hCTLA4-hCD59) was expressed in PAEC and fibroblasts derived from HT-transgenic pigs (Costa et al., 1999). HT downregulates Galα1,3-Gal antigen expression and generates fucosylated residues (H-antigen, the O blood group antigen) that are universally tolerated in humans (Costa et al., 1999; Sandrin et al., 1995). Co-expression of high levels of HT and the present chimeric molecule led to maximal protection from human NK cell-mediated cytotoxicity. These results demonstrate a role for porcine CD86 in triggering human NK cell activation. Strategies that combine inhibition of CD86 with carbohydrate remodeling have the potential to overcome DXR and improve the prospects of clinical xenotransplantation.

Chimeric proteins capable of conferring resistance to humoral and cellular mechanisms of immune attack do not require that the domains with each activity are directly attached to one another. For example, the amino acid sequence having C5b-9 inhibitory activity and the amino acid sequence having T-cell inhibitory and NK cell inhibitory activity do not have to be directly attached to one another. A linker sequence can separate these two sequences. The linker preferably comprises between about one and at least about 6 amino acid residues. Suitable linker sequences can include glycines. Other amino acids, as well as combinations of amino acids, can be used in the linker region if desired. In one embodiment (pCC), amino acids 153 to 158 of SEQ ID NO:2 (GGGGGG (SEQ ID NO:12)) are the linker sequence. In another embodiment (hCC), amino acids 152 to 157 of SEQ ID NO:4 (ASASAS (SEQ ID NO:13)) are the linker sequence. These molecules are designed for cell surface expression attached via the CD59 GPI anchor.

Further embodiments provide nucleic acids, e.g., recombinant cDNAs, encoding chimeric proteins capable of conferring resistance to humoral and cellular mechanisms of immune attack. These nucleic acids may include exons encoding specific inhibitory peptides.

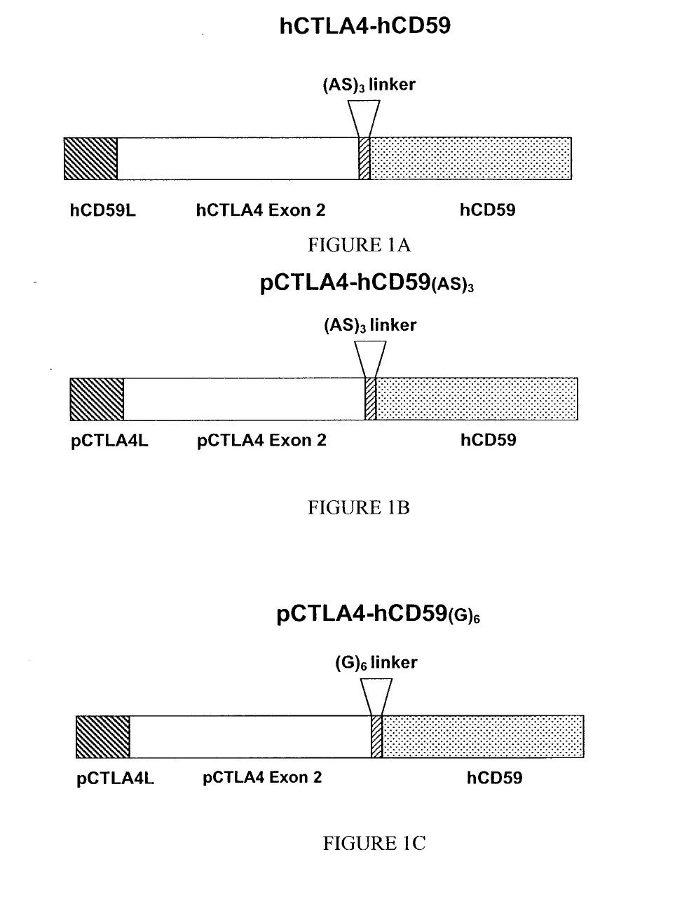

One such embodiment provides recombinant cDNA which includes an exon of the human homologue of CTLA4 inserted into the coding region of human CD59, bisecting CD59 between the leader peptide and the mature peptide post-translational processing site (see FIG. 1A and SEQ ID NOs:3-4). A further embodiment provides recombinant cDNA which includes an exon of the porcine homologue of CTLA4 inserted into the coding region of human CD59, bisecting CD59 between the leader peptide and the mature peptide post-translational processing site (see SEQ ID NO:2). More preferred embodiments provide recombinant cDNAs which include an exon of the porcine homologue of CTLA4 with its own leader sequence followed by a (G)₆ or (AS)₃ linker and the coding region of human CD59 (see FIGS. 1B-C and SEQ ID NOs:24-27). In each embodiment, the cDNA may include a coding sequence for a putative GPI anchor linkage site corresponding to amino acid 235 of pCC (as shown in SEQ ID NO:2) or 234 of hCC (as shown in SEQ ID NO:4) and amino acid 77 of mature CD59 (as shown in SEQ ID NO:10).

Molecules comprising nucleotide sequences encoding the CTLA4 and CD59 or DAF domains can be prepared using a variety of techniques known in the art. For example, the exon 2 sequences encoding CTLA4 (nucleotide numbers 112-483 of SEQ ID NO:7) and CD59 leader peptide region (nucleotides 80-154 of SEQ ID NO:9) and mature peptide nucleotide (nucleotides 155-463 of SEQ ID NO:9) domains can be produced using PCR generation and/or restriction digestion of cloned genes to generate fragments encoding amino acid sequence having T-cell or NK cell and C5b-9 inhibitory activities. These fragments can be assembled using PCR fusion or enzymatic ligation of the restriction digestion products (Sambrook et al., 1989; Ausubel et al., 1991). Alternatively, any or all of the nucleic acid fragments used to assemble the chimeric genes can be synthesized by chemical means. In another embodiment, the nucleotide sequences encoding the CTLA4 and DAF domains can be produced using PCR generation and/or restriction digestion of cloned genes to generate fragments encoding amino acid sequences having T-cell or NK cell and C3 inhibitory activities. These fragments also can be assembled using PCR fusion or enzymatic ligation of the restriction digestion products (Sambrook et al., 1989; Ausubel et al., 1991). Any or all of the nucleic acid fragments used to assemble these chimeric genes can be synthesized by chemical means as well.

In another embodiment, recombinant expression vectors which include nucleic acid fragments of the chimeric protein are provided. The nucleic acid molecule coding for such a chimeric protein can be inserted into an appropriate expression vector, i.e., a vector that contains the necessary elements for the transcription and translation of the inserted protein-encoding sequence. Suitable host vector systems include, but are not limited to, mammalian cell systems infected with virus (e.g., vaccinia virus, adenovirus, retroviruses, etc.); mammalian cell systems transfected with plasmids; insect cell systems infected with virus (e.g., baculovirus); microorganisms such as yeast containing yeast expression vectors, or bacteria transformed with bacteriophage DNA, plasmid DNA or cosmid DNA (see, for example, Goeddel, 1990). Commonly used promoters and enhancers derived from polyoma virus, adenovirus, Simian virus 40 (SV40), the Molony murine leukemia virus (MMLV), including the long terminal repeat (MMLV-LTR), and human cytomegalovirus (CMV), including the cytomegalovirus immediate-early gene 1 promoter and enhancer are suitable. Suitable eukaryotic promoters include P-actin (Ng et al., 1989) and H2Kb (Fodor et al., 1994).

In a preferred embodiment, the cDNA of interest is cloned into a retroviral vector that is subsequently transfected into a mouse cell line called a “packaging cell line”. The manipulation of retroviral nucleic acids to construct retroviral vectors and packaging cells is accomplished using techniques known in the art. See, for example, Ausubel et al., 1991 (Volume 1, Section III, units 9.10.1 to 9.14.3); Sambrook et al., 1989; Miller and Buttimore, 1986; Eglitis and Anderson, 1988; U.S. Pat. Nos. 4,650,764; 4,861,719; 4,980,289; 5,112,767; and 5,124,263; as well as PCT Patent Publications Nos. WO 85/05629; WO 89/07150; WO 90/02797; WO 90/02806; WO 90/13641; WO 92/05266; WO 92/07943; WO 92/14829; and WO 93/14188. Typically, the retroviral vector contains a gene that allows for selection via resistance to drugs such as puromycin. It also contains nucleic acid sequence that allows for random or directed integration of the vector into a eukaryotic genome. Drug resistant cell lines are selected. These cells will produce virus particles capable of infecting other cell lines. Porcine aortic endothelial cells (PAECs) are infected with the viruses by a process called viral transduction. The transduced PAECs are selected for by drug resistance. Drug resistant cells contain an integrated copy of the viral vector DNA. Once in the porcine genome, vector sequences or sequences associated with the chimeric gene control the expression of the chimeric protein.

In particular, the retroviral vectors of the invention can be prepared and used as follows. First, a retroviral vector containing nucleic acid encoding for the chimeric protein described herein above is constructed and packaged into transducing viral particles (virions) using an amphotropic packaging system, preferably one suitable for use in gene therapy applications. Examples of such packaging systems are found in, for example, Miller and Buttimore, 1986; Markowitz et al., 1988; Cosset et al., 1990; U.S. Pat. Nos. 4,650,764; 4,861,719; 4,980,289; 5,112,767; and 5,124,263; and PCT Patent Publications Nos. WO 85/05629; WO 89/07150; WO 90/02797; WO 90/02806; WO 90/13641; WO 92/05266; WO 92/07943; WO 92/14829; and WO 93/14188. A preferred packaging cell is the PA317 packaging cell line (ATCC CRL 9078, Manassas, Va.). The generation of “producer cells” is accomplished by introducing retroviral vectors into the packaging cells. The producer cells generated by the foregoing procedures are used to produce the retroviral vector particles (virions). This is accomplished by culturing of the cells in a suitable growth medium. Preferably, the virions are harvested from the culture and administered to the target cells which are to be transduced. Examples of such retroviral vectors are found in, for example, Korman et al., 1987; Morgenstern and Land, 1990; U.S. Pat. Nos. 4,405,712; 4,980,289; and 5,112,767; and PCT Patent Publications Nos. WO 85/05629; WO 90/02797; and WO 92/07943. A preferred retroviral vector is the MMLV derived expression vector pLXSN (see Miller et al., 1989) or pBABEpuro (Morgenstern and Land, 1990). DNA can be introduced into cells by any standard method of transfection such as polybrene, DEAE, calcium phosphate, lipofection, electroporation (see Sambrook et al., 1989).

Engineered transgenic animals (for example, rodent, e.g., mouse, rat capybara, and the like; lagomorph, e.g., rabbit, hare, and the like; ungulate, e.g., pig, cow, goat, sheep, and the like; etc.) that express the chimeric protein described herein on the surfaces of their cells are provided using any suitable techniques known in the art. These techniques include, but are not limited to, microinjection, e.g., of pronuclei; electroporation of ova or zygotes; nuclear transplantation; and/or the stable transfection or transduction of embryonic stem cells derived from the animal of choice.

A common element of these techniques involves the preparation of a transgene transcription unit. Such a unit includes a DNA molecule which generally includes: 1) a promoter, 2) the coding sequence of the transgene, and 3) a polyadenylation signal sequence. Other sequences, such as, enhancer, insulator and intron sequences, can optionally be included. The unit can be conveniently prepared by isolating a restriction fragment of a plasmid vector which expresses the CTLA4-CD59 protein in, for example, mammalian cells. Preferably, the restriction fragment is free of bacterially derived sequences that are known to have deleterious effects on embryo viability and gene expression.

The most well known method for making transgenic animals is that used to produce transgenic mice by superovulation of a donor female, surgical removal of the egg, injection of the transgene transcription unit into the pro-nuclei of the embryo, and introduction of the transgenic embryo into the reproductive tract of a pseudopregnant host mother, usually of the same species. See, for example, U.S. Pat. No. 4,873,191; Brinster et al., 1985; Hogan et al., 1986; Robertson, 1987. The use of this method to make transgenic livestock is also widely practiced by those of skill in the art. As an example, transgenic swine are routinely produced by the microinjection of a transgene transcription unit into pig embryos. See, for example, PCT Publication No. WO 92/11757. In brief, this procedure may, for example, be performed as follows. First, the transgene transcription unit is gel isolated and extensively purified through, for example, an ELUTIP column (Schleicher & Schuell, Keene, N.H.), dialyzed against pyrogen free injection buffer (10 mM Tris, pH 7.4, 0.1 mM EDTA in pyrogen free water) and used for embryo injection. Embryos are recovered from the oviduct of a hormonally synchronized, ovulation induced sow, preferably at the pronuclear stage. They are placed into a 1.5 mL microfuge tube containing approximately 0.5 mL of embryo transfer media (phosphate buffered saline with 10% fetal calf serum). These are centrifuged for 12 minutes at 16,000×g in a microcentrifuge. Embryos are removed from the microfuge tube with a drawn and polished Pasteur pipette and placed into a 35 mm petri dish for examination. If the cytoplasm is still opaque with lipid such that the pronuclei are not clearly visible, the embryos are centrifuged again for an additional 15 minutes. Embryos to be microinjected are placed into a drop of medium (approximately 100 μL) in the center of the lid of a 100 mm petri dish. Silicone oil is used to cover this drop and to fill the lid to prevent the medium from evaporating. The petri dish lid containing the embryos is set onto an inverted microscope equipped with both a heated stage (37.5° C.-38° C.) and Hoffman modulation contrast optics (200× final magnification). A finely drawn and polished micropipette is used to stabilize the embryos while about 1-2 picoliters of injection buffer containing approximately 200-500 copies of the purified transgene transcription unit is delivered into the nucleus, preferably the male pronucleus, with another finely drawn and polished micropipette. Embryos surviving the microinjection process as judged by morphological observation are loaded into a polypropylene tube (2 mm inner diameter) for transfer into the recipient pseudopregnant sow. Offspring are tested for the presence of the transgene by isolating genomic DNA from tissue removed from the tail of each piglet and subjecting about 5 micrograms of this genomic DNA to nucleic acid hybridization analysis with a transgene specific probe. In a preferred embodiment, transgenic animals are produced according to the methods disclosed in PCT Publication No. WO 99/07829, the contents of which are incorporated herein by reference.

Another commonly used technique for generating transgenic animals involves the genetic manipulation of embryonic stem cells (ES cells) as described in PCT Publication No. WO 93/02188 and Robertson (1987). In accordance with this technique, ES cells are grown as described in, for example, Robertson (1987) and in U.S. Pat. No. 5,166,065 to Williams et al. Genetic material is introduced into the embryonic stem cells by, for example, electroporation according, for example, to the method of McMahon and Bradley (1990), or by transduction with a retroviral vector according, for example, to the method of Robertson et al. (1986), or by any of the various techniques described in Robertson (1987). Chimeric animals are generated as described, for example, in Bradley (1987). Briefly, genetically modified ES cells are introduced into blastocysts and the modified blastocysts are then implanted in pseudo-pregnant female animals. Chimeras are selected from the offspring, for example by the observation of mosaic coat coloration resulting from differences in the strain used to prepare the ES cells and the strain used to prepare the blastocysts, and are bred to produce non-chimeric transgenic animals.

Other methods for the production of transgenic animals are disclosed in U.S. Pat. No. 5,032,407, PCT Publication No. WO 90/08832, and Bondioli et al. (2001).

In order that those skilled in the art may be better able to practice the compositions and methods described herein, the following examples are given as an illustration of the preparation of chimeric proteins having both complement inhibitory domains and T-cell and NK cell inhibitory domains, as well as their ability to be expressed on a cell surface of an antigen presenting cell, bind targets on the same antigen presenting cell, and exhibit T-cell and NK cell inhibitory activity. It is to be understood that commercially available reagents and/or instrumentation referred to in the examples were used according to the manufacturer's instructions unless otherwise indicated.

### EXAMPLE 1

### Construction of Human CTLA4-Human CD59 Chimeric Molecules

A full length human CTLA4 cDNA was isolated from human peripheral blood leukocytes (PBLs) that were activated with 3 ng/mL phorbol 12 myristate 13 acetate (PMA) and 0.4 μg/mL ionomycin (commercially available from Sigma, St. Louis, Mo.). First strand cDNA synthesized from PBL RNA using reverse transcriptase as recommended by the vendor (Seikagaku America, Inc., Rockville, Md.) was used as a template in a polymerase chain reaction (PCR) (Mullis et al., 1986) to amplify the extracellular domain encoded by exon 2 (according to methods described in Brunet et al. (1987), Brunet et al. (1988) and Dariavach et al. (1988)). The 5′ forward oligonucleotide 5′-GCCTGCAGATGCACGTGGCC-3′ (SEQ ID NO:14) and the 3′ reverse oligonucleotide 5′-GGCTGCAGGGAGGCGGAGGCGGAGGCGTCAGAATCTGG-3′ (SEQ ID NO:15), which contained homologous nucleotides and nucleotide encoding linker sequence, were used in the following PCR reaction mixture to amplify a CTLA4 DNA fragment (bases 112-483 of SEQ ID NO:7) and to add a 6 amino acid bridge (ASASAS (SEQ ID NO:13)) and PstI sites for cloning. Five microliters of a first strand synthesis of cDNA made from activated PBLs was amplified in the presence of 10 mM magnesium chloride, 500 μM dNTPs, 2 μM oligonucleotides, 2.5 Units Taq polymerase (Perkin Elmer, Norwalk, Conn.) for forty cycles. Each cycle consisted of denaturing for one minute at 95° C., annealing at 55° C. for one minute, and polymerizing at 72° C. for one minute. One cycle of polymerization at 72° C. for ten minutes insured the addition of thymidine overhang for TA cloning. The CTLA4 exon 2 fragment was ligated into the pCRII.1TOPO vector using the TOPO TA cloning kit (commercially available from Invitrogen, Carlsbad, Calif.) and used to transform the TOP 10 strain of E. coli (commercially available from Invitrogen, Carlsbad, Calif.). Plasmids containing the appropriately sized fragment were isolated and the inserts were subjected to DNA sequencing to confirm the integrity and identity of the DNA (Wm. Keck Foundation Biotechnology Resource Laboratory, Yale University, New Haven, Conn.). Plasmids that contained the verified CTLA4 exon 2 insert were digested with PstI and the CTLA4 exon 2 fragment was isolated. A GEM7Z plasmid (commercially available from Clontech, Palo Alto, Calif.) that contained the human CD59 sequence that has a unique PstI site located between the human CD59 signal sequence and mature protein coding sequence was digested with PstI. The PstI fragment that contained CTLA4 exon 2 was ligated into the corresponding PstI site on GEM7Z and plasmids that contained the correct insert were selected. A BamHI-EcoRI fragment containing the entire chimeric human CTLA4-human CD59 (hCTLA4-hCD59) gene was excised from the plasmid and then subcloned into the amphotropic retroviral expression vector pBABEpuro (Morgenstern and Land, 1990) to generate the expression vector hCTLA4-hCD59BABEpuro.

### EXAMPLE 2

### Cloning of Porcine CTLA4

Porcine cDNA was prepared from porcine PBLs that were activated with 3 ng/mL PMA and 0.4 μg/mL ionomycin (commercially available from Sigma, St. Louis, Mo.). The cDNA was used as a template in a PCR using redundant primers designed from a comparison of human (GenBank Accession No. NM_{—}005214), mouse (GenBank Accession No. X05719), rabbit (GenBank Accession No. D49844) and bovine (GenBank Accession No. X93305) CTLA4 nucleotide sequences. The 5′ forward oligonucleotide: 5′-CCCMYMMAGCCATGGCTYGYYYYGG-3′ (SEQ ID NO:16) together with the 3′ reverse oligonucleotide: 5′-CCTCARTTRATRGGAATAAAATAAGGCTG-3′ (SEQ ID NO:17) were used in PCR conditions as described in Example 1, except annealing occurred at 45° C., and twenty cycles of amplification were used. The PCR produced a 672 base pair fragment that was cloned into the TOPO vector using the TOPO TA cloning kit (commercially available from Invitrogen, Carlsbad, Calif.). DNA sequence analysis confirmed that the insert was the full-length porcine CTLA4 clone (SEQ ID NO:5).

### EXAMPLE 3

### Construction of Porcine CTLA4-Human CD59 Chimeric Molecules

The extracellular domain of porcine CTLA4 encoded by exon 2 was PCR amplified from the TOPO plasmid prepared in Example 2 using a 5′ forward oligonucleotide: 5′-CCATGCATATGCACGTGGCCCAGCCTGCAG-3′ (SEQ ID NO:18) and a 3′ reverse oligonucleotide: 5′-CATGCATGCCACCGCCACCGCCACCGAAATCAGAATCTGGGCAT GGTTCTGGATCAATG-3′ (SEQ ID NO:19) that contained homologous pCTLA4 sequence, restriction sites and linker sequence using the same PCR conditions as described in Example 1, except that only 35 cycles of amplification were used to generate a 408 base pair DNA fragment (including 375 base pairs of pCTLA4 exon 2, 18 base pairs of linker sequence, and tails of 8 and 7 base pairs). The fragment was cloned into the TOPO vector using the TOPO TA cloning kit (commercially available from Invitrogen, Carlsbad, Calif.). DNA sequence analysis confirmed that the insert was the porcine CTLA4 exon 2. The plasmid was digested with NsiI and the fragment that contained the CTLA4 exon 2 was isolated and ligated into the PstI site of the GEM7Z plasmid that contained human CD59 as described in Example 1. The BamHI-EcoRI fragment containing the chimeric pig CTLA4-human CD59 molecule (pCTLA4-hCD59) was excised from the plasmid and then subcloned into the amphotropic retroviral expression vector pBABEpuro (see Morgenstern and Land, 1990) to generate the expression vector pCTLA4hCD59BABEpuro. This construction with a human CD59 leader sequence resulted in very low expression in PAECs.

To improve the expression of pCTLA4hCD59, the human signal sequence of CD59 was replaced with the native porcine signal sequence of pCTLA4. This was performed both with a 124 amino acid plus AlaSerAlaSerAlaSer (SEQ ID NO:13) bridge version and with a 125 amino acid plus GlyGlyGlyGlyGlyGly (SEQ ID NO:12) bridge version of pCC (porcine CTLA4humanCD59). For the 124 amino acid version, 5′ forward primer 5′-CCATGCATATGCACGTGGCCCAGCCTGC-3′ (SEQ ID NO:22) and 3′ reverse primer 5′-CCATGCATGGAGGCGGAGGCGGAGGCATCAGAATCTGGGCATGGTTCTGG-3′ (SEQ ID NO:23) were used to amplify exon 2, nucleotides 112-483 of SEQ ID NO:5, adding amino acid bridge AlaSerAlaSerAlaSer (SEQ ID NO:13) and flanking NsiI sites using the full length clone as a template. This was cloned by TOPO TA cloning and the NsiI fragment was prepared and subcloned into the PstI site of hCD59GEM7Z between the human CD59 leader and the mature human CD59 protein sequence thereby producing a clone with a human CD59 leader, 124 amino acids of porcine CTLA4 plus the AlaSerAlaSerAlaSer (SEQ ID NO:13) bridge, and mature human CD59 protein sequence. The human CD59 leader and part of the pCTLA4 were removed from this clone by digestion with BamHI (a BamHI site is present at bases 44-49 of SEQ ID NO:9, which site is 5′ of the human CD59 leader sequence), Klenow treatment, HincII digestion (a HincII site is present at bases 282-287 of SEQ ID NO:5, which site is within exon 2 of pCTLA4) and dephosphorylation with calf intestinal alkaline phosphatase. The deleted fragment was replaced with the EcoRV-HincII fragment of the full length clone containing the porcine native leader and part of exon 2. The EcoRV restriction site is located in the pCRII.1 TOPO multicloning site into which the 672 base pair pCTLA4 was TOPO TA cloned. This was cleaved with EcoRI (EcoRI sites are located in the multicloning site of the pCRII.1 TOPO vector and at bases 471-476 of the human CD59 SEQ ID NO:9) to yield an 840 base pair fragment (SEQ ID NO:24 which includes the complete EcoRI site at each end; FIG. 1B) that was cloned into pBABEpuro. This clone induced high expression in Balb/3T3 cell line #22.

For the 125 amino acid version, a 5′ forward primer 5′-CCATGCATATGCACGTGGCCCAGCCTGCAG-3′ (SEQ ID NO:18) and 3′ reverse primer 5′-CATGCATGCCACCGCCACCGCCACCGAAATCAGAATCTGGGCATGGTTCTGGATCA ATG-3′ (SEQ ID NO:19) were use to amplify exon 2 nucleotides 112-486 of SEQ ID NO:5, thereby adding amino acid bridge GlyGlyGlyGlyGlyGly (SEQ ID NO:12) and flanking NsiI sites using the full length clone. This was TOPO TA cloned into pCRII.1 TOPO (Invitrogen, Carlsbad, Calif.). The NsiI fragment was isolated and subcloned into the PstI site of hCD59GEM7Z (described in Example 1). The human CD59 leader and part of the pCTLA4 exon 2 were removed by digestion with BamHI, Klenow treatment, HincII digestion and dephosphorylation with calf intestinal alkaline phosphatase. This was replaced with the EcoRV-HincII fragment of the full length clone containing the native pCTLA4 leader and part of exon 2. This was digested with EcoRI to yield an 843 base pair fragment (SEQ ID NO:26 which shows the complete EcoRI site at both ends; FIG. 1C) that was cloned into pBABEpuro. This construction produced high expression in Balb/c 3T3 cell line #54.

### EXAMPLE 4

### Soluble Porcine CTLA4

Soluble porcine CTLA4 was generated by subcloning exons 1 and 2 into the APEX 3 (Evans et al., 1995) mammalian expression vector HuIgG1fcAPEX 3. A 5′ forward oligomer (5′-GGGCTAGCAGCCATGGCTCGTTTCGGATTCCGG-3′ (SEQ ID NO:20)) and a 3′ reverse oligomer (5′-CGGATCCGAAATCAGAATCTGGGCATGGTTCTGGATCAATGAC-3′ (SEQ ID NO:21)) were used to amplify exons 1 and 2 from nucleotides 1-486 of SEQ ID NO:5, adding a 5′ NheI site and a 3′ BamHI site for ease of cloning into APEX. The NheI-BamHI insert was ligated to a 9831 base pair XbaI-BamHI APEX 3 vector. This vector uses a CMV promoter to translate the insert in frame with 927 nucleotides of human IgG1 heavy chain for secretion as a human IgG1 fusion protein.

### EXAMPLE 5

### Isolation and Culture of Porcine Cells

PAEC and fibroblasts from HT-transgenic and non-transgenic littermate pigs were isolated as described (Costa et al., 1999). Briefly, primary cultures of PAEC were obtained by scraping the aorta with a scalpel and culturing in DMEM 10% FCS. Primary cultures of fibroblasts were obtained by mincing ear tissue, or lung tissue in the case of HTAT21F₁ fibroblasts, and culturing in DMEM 10% FCS. PAEC and fibroblasts were cultured in DMEM 10% FCS supplemented with Endothelial mitogen (EM, 50 mg/L) (Biomedical Technologies, Inc., Stoughton, Mass.) for expansion, but the EM was withdrawn 24-48 hours before the cytotoxicity assays to avoid interference.

### EXAMPLE 6

### hCTLA4-hCD59 PAEC Cell Lines

To create a cell line that expresses the human CTLA4-humanCD59 chimeric molecule, a cell line must be created that produces retroviruses that contain the necessary gene. Another cell line must then be transduced with the virus to create a population of cells that express the protein. To produce the retrovirus, the murine amphotropic packaging cell line PA317 (ATCC, Manassas, Va.) was transfected with the expression vector prepared in Example 1 (hCTLA4hCD59BABEpuro) or pBABEpuro (vector control DNA) by the polybrene method (see Sambrook et al., 1989). Ten micrograms of DNA were added to PA317 cells in 5 mL of Dulbecco's minimum essential medium (DMEM), available from Cellgro, Herndon, Va., containing 10% heat inactivated fetal bovine serum (FBS) followed by a five hour treatment with 30 μg/mL polybrene (Sigma, St. Louis, Mo.) without a dimethylsulfoxide (DMSO) shock. The cells were washed and incubated in DMEM with 10% FBS and 48 hours post transfection the cells were treated with 5 μg/mL puromycin to select drug resistant transfectants. The transfected PA317 cells produced retrovirus and viral supernatants which were harvested as described by Morgenstern and Land (1990). The next step was to transduce porcine cells to create a porcine cell line that expresses human CTLA4-human CD59 protein or the porcine CTLA4-hCD59. Using standard methods 5×10⁵ porcine aortic endothelial cells (PAEC) or porcine fibroblasts were transduced using 1.5 mL of viral supernatant added to 3.5 mL of DMEM with 10% FBS followed by the addition of polybrene to 8 μg/mL for 5 hours at 37° C. Following transduction the cells were incubated in DMEM with 10% FBS for 48 hours. The cells were split into selection medium (DMEM with 10% FBS and 3 μg/mL puromycin for PAECs or 4 μg/mL puromycin for fibroblasts).

Puromycin resistant cell populations that express human CTLA4-human CD59 chimeric molecules were identified by fluorescence activated cell sorting (FACS) analysis using antibodies to human CTLA4 and human CD59 using standard methodologies (Coligan et al., 1992).

### EXAMPLE 7

### pCTLA4-hCD59 Cell Lines

Production of a PAEC line to express pCTLA4hCD59 was carried out in the same manner as described in Example 6, except that the DNA used to transfect the virus producing cell line was the expression vector pCTLA4hCD59BABEpuro prepared in Example 3.

### EXAMPLE 8

### Porcine CTLA4-Human CD59 Balb/c 3T3 Cell Lines

Porcine CTLA4-human CD59 Balb/c 3T3 cell lines were generated by direct transfection of Balb/c 3T3 (ATCC, Manassas, Va.) using 2 μg DNA, and 0.025 mL LipofectAmine diluted in 1 mL of Optimem (Life Technologies, Gaithersburg, Md.) on 6×10⁵ cells per well of a 6 well plate (Becton Dickinson, Franklin Lakes, N.J.). The cells were transfected for 4 hours at 37° C. Following aspiration of transfection mixture, the cells were washed and fed with DMEM medium containing 10% heat inactivated calf serum (Summit Biotechnology, Ft. Collins, Colo.). After forty-eight hours, transfectants were cultured in complete medium containing 3 μg/mL puromycin as above. Cell lines were cloned for high expression and analyzed by FACS (Becton Dickinson, Franklin Lakes, N.J.) for the chimeric molecules as described below.

### EXAMPLE 9

### Jurkat Cells

Jurkat cells (ATCC, Manassas, Va.) were cultured in complete RPMI (Mediatech, Herndon, Va.) containing 1 mM β-mercaptoethanol (Sigma, St. Louis, Mo.). Soluble porcine CTLA4Ig was secreted from EBNA (Epstein Barr nuclear antigen) 293 cells (Invitrogen, Carlsbad, Calif.) that were transfected with the soluble pCTLA4-human IgG1fc APEX 3 construct (see Example 4) and selected using 300 U/mL hygromycin and 250 μg/mL neomycin.

### EXAMPLE 10

### Expression of Human CD59 and Human or Porcine CTLA4

Drug resistant populations of transfected PAEC or Balb/c 3T3 were assayed for expression of human CD59 and human or porcine CTLA4. Briefly, cells were incubated with one of the following: 5 μg/mL rabbit anti-porcine B7.1 (Cocalico, Reamstown, Pa.) or rabbit anti-histidine (Santa Cruz Biotechnology Inc., Santa Cruz, Calif.); 10 μg/mL rat anti-mouse B7.1 (Pharmingen, San Diego, Calif.), mouse anti-porcine B7.2 clone 10-11-34 (Alexion, Cheshire, Conn.), mouse anti-human CD152 (Ancell Corp., Bayport, Minn.), rabbit anti-human CD59 ALP-3 (Cocalico Reamstown, Pa.), mouse anti-hCD59 clone MEM-43 or clone BRA10G (BIODESIGN International, Kennebunk, Me.); 20 μg/mL rabbit anti-pCTLA4 ALP-67 (Cocalico, Reamstown, Pa.), or 50 μg/mL soluble porcine B7.1-hist tagged (Alexion, Cheshire, Conn.), in 0.1 mL of Dulbecco's PBS (DPBS) containing 1% FBS for 30 minutes at 4° C. Cells were washed with DPBS before incubation with FITC conjugated antibodies to mouse or rabbit IgG (Zymed, So. San Francisco, Calif.). In some assays cells were treated with PI-PLC (Boehringer Mannheim GmbH, Indianapolis, Ind.) at 1 U/mL in serum free DPBS for 1 hour at 37° C. prior to FACS analysis on a Becton Dickinson FACSort (Becton Dickinson, San Jose, Calif.).

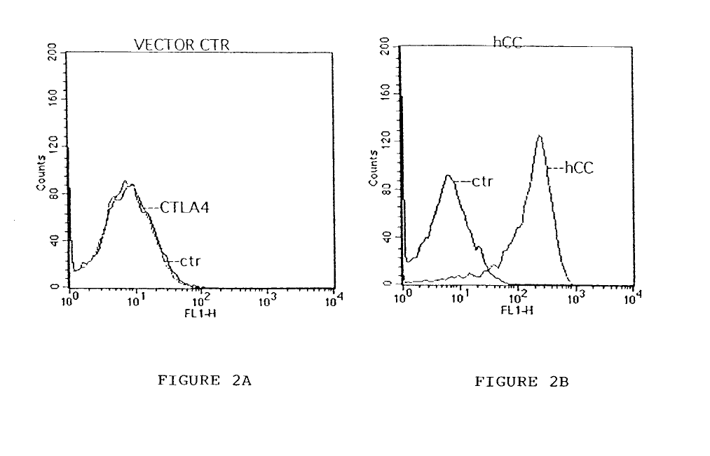

Drug resistant populations of PAEC (single clones) or porcine fibroblasts (from pools of 3-5 colonies) transduced with hCTLA4hCD59BABEpuro or pBABEpuro vector alone were assayed for cell surface expression of CD59 and CTLA4 by FACS analysis using antibodies to human CTLA4 and human CD59. Briefly, cells were incubated with 10 μg/mL anti-CTLA4 (ANC152.2 commercially available from Ancell, Bayport, Minn.) for 30 minutes at 4° C. in 0.1 mL of Dulbecco's phosphate buffered saline (DPBS) containing 1% FBS or bovine serum albumin (BSA). Cells were washed with DPBS before incubation with FITC conjugated antibodies to mouse IgG (commercially available from Zymed, So. San Francisco, Calif.). FACS analysis was carried out on a Becton Dickinson FACSORT (Becton Dickinson, Franklin Lakes, N.J.) instrument using standard methodologies (Coligan et al., 1992). The pBABEpuro transduced cells were negative for hCTLA4 expression (FIG. 2A). The anti-CD59 polyclonal antibody ALP3 showed slight background reactivity with the pBABEpuro transduced PAECs. The hCTLA4hCD59 PAECs exhibited high level expression of hCTLA4 and hCD59 as determined by FACS analysis with antibodies specific to each moiety (FIG. 2B).

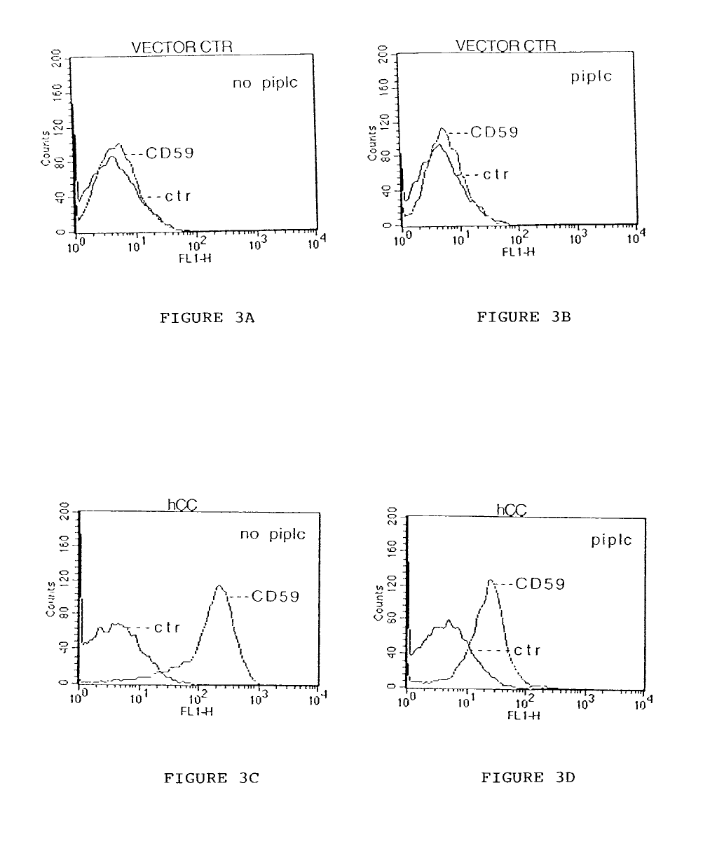

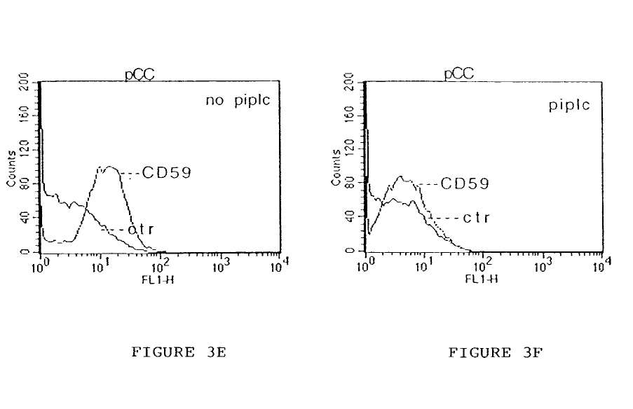

The hCTLA4hCD59 cell line was also treated with PI-PLC and then assayed for expression of the chimeric molecule to further demonstrate that the chimeric molecules were anchored to the cell surface with a CD59 GPI anchor linkage, by enzymatically cleaving the CD59 GPI membrane attachment. Phosphatidyl inositol phospholipase C (PI-PLC) enzymatically cleaves GPI-linked molecules from the surface of cells and therefore should cleave the hCTLA4-hCD59 molecule from the transduced PAECs. FIGS. 3A-D illustrate the loss of cell surface expression following PI-PLC treatment, as indicated by reduced antibody reactivity following enzymatic digestion. FACS analysis was performed as described above for FIGS. 2A-B with the following exceptions. Drug resistant populations of PAECs transduced with pBABEhCTLA4-hCD59 or pBABE vector alone were assayed for expression of CD59. Briefly, cells were incubated with 10 μg/mL of the anti-CD59 antibody for 30 minutes at 4° C. in 0.1 mL of DPBS containing 1% FBS or BSA. Cells were washed with DPBS before incubation with FITC conjugated antibodies to mouse IgG (Zymed, So. San Francisco, Calif.). In addition, an aliquot of the cell lines was treated with PI-PLC (Boehringer Mannheim GmbH, Indianapolis, Ind.) at 1 U/mL for 1 hour at 37° C. prior to antibody incubations and FACS analysis on a Becton Dickinson FACSORT (Becton Dickinson, Franklin Lakes, N.J.).

FACS analysis for cell surface expression of pCTLA4-hCD59 in PAEC cells was carried out as described above for the hCTLA4-hCD59. However, detection of the pCTLA4 had to be accomplished by a different method because the human specific anti-CTLA4 antibody, ANC152.2, only bound to the human CTLA4 molecule and did not crossreact to the pig molecule. Therefore, the pCTLA4-hCD59 molecule was detected with the anti-CD59 mAb BRA-10G or MEM43 (BIODESIGN International, Kennebunk, Me.) using the same methods as described above for ANC152.2. FIG. 3E illustrates CD59 expression on pCTLA4-hCD59 transduced PAEC. The pCTLA4hCD59 cell line was also treated with PI-PLC and then assayed for expression of the chimeric molecule to demonstrate that the chimeric molecules were anchored to the cell surface with a CD59 GPI anchor linkage by cleaving the CD59 GPI membrane attachment. FIG. 3F illustrates the loss of cell surface expression following PI-PLC treatment. Both moieties could not be detected post digestion.

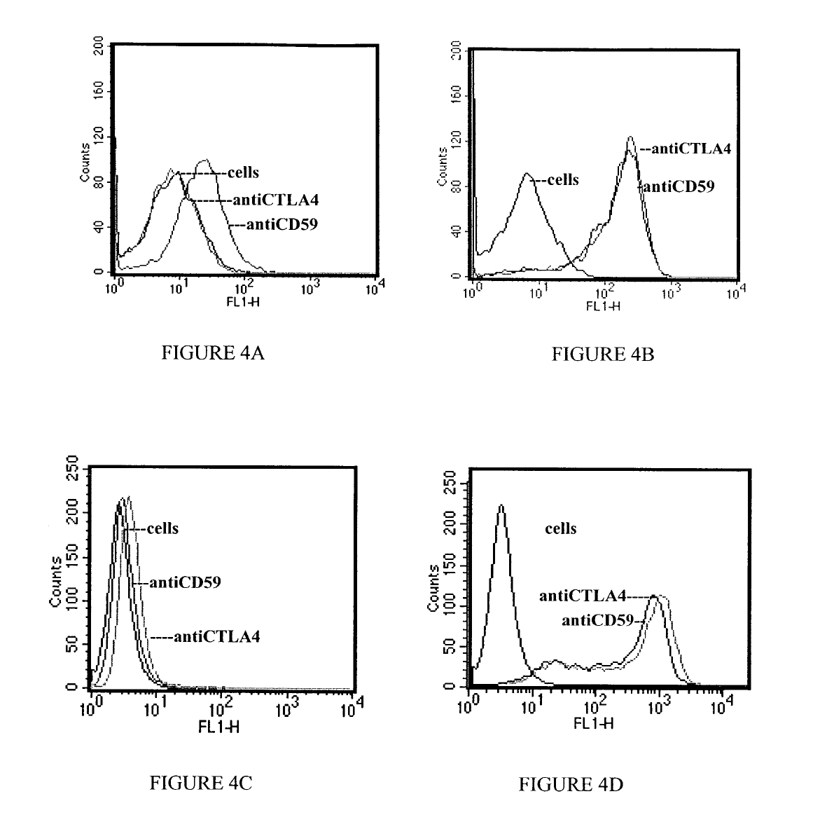

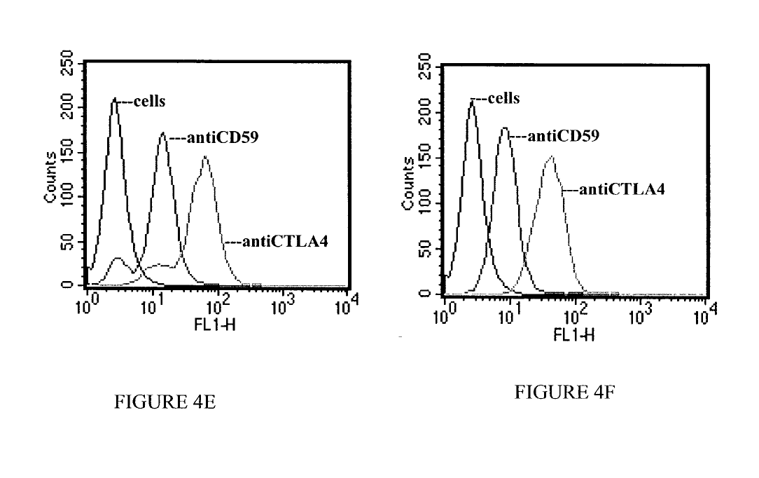

Balb/3T3 transfected cells were analyzed by flow cytometry. The vector alone transfected cells were negative for both CD59 and CTLA4 (FIG. 4A). The hCTLA4-hCD59 clone 29 had very high level expression as determined by BRA10G and ANC152.2 antibody reactivity (FIG. 4B). Two clones, pCTLA4(AS)₃-CD59-22 and pCTLA4(G)₆-hCD59-54, expressing the porcine versions of the chimeric molecules with the porcine CTLA4 leader sequence were characterized using ALP-67 which is a polyclonal antibody to pCTLA4 and using BRA10G which is a monoclonal antibody to hCD59. The human specific anti-CTLA4 antibody ANC152.2 only bound to the human CTLA4 molecule and did not cross react to the pig molecule. Both porcine CTLA4-hCD59 constructs were expressed on the mouse cell surface as determined by anti-pCTLA4 and anti-CD59 antibody reactivity. The expression of both porcine constructs was consistently lower than the human CTLA4-CD59 (FIGS. 4B, D-F). The different levels of detection, i.e., anti-CTLA4 vs. anti-CD59, observed in the pCTLA4-hCD59 expressing cells is most likely due to the difference in antibody affinity between the monoclonal and the polyclonal anti-sera.

Antibody reactivity to the hCTLA4-hCD59 expressed protein, regardless of the host cell type, was consistent between anti-CD59 antibodies (BRA10G and ALP3) and the anti-CD152 (anti-CTLA4) human antibody, ANC152.2 (FIGS. 4B, D). However, neither of the two porcine versions expressed on Balb/3T3 cells exhibited equal antibody reactivity (FIGS. 4E-F).

### EXAMPLE 11

### Complement Killing Assays

Five thousand vector control or 1×10⁴ hCTLA4-CD59 cells were seeded into the wells of a flat bottom 96 well plate 24 hours ahead of time. Adherent cells were washed twice using Hank's balanced salt solution, HBSS (Mediatech Inc., Herndon, Va.) containing 1% bovine serum albumin (Sigma, St. Louis, Mo.). Cells were sensitized by incubating with a polyclonal anti-PAEC antibody (Cocalico, Reamstown, Pa.) or polyclonal anti-mouse antibody (Joe Madre, Yale University, New Haven, Conn.), followed by incubation with the intracellular dye, Calcein AM (Molecular Probes, Eugene, Oreg.) in HBSS/BSA for 30 minutes at 37° C. Excess Calcein AM was removed with two additional washes. Normal human serum (Sigma, St. Louis, Mo.) was used as a complement source and was added to a final concentration of 10, 20, or 40% in 0.05 mL volume diluted in HBSS and the cells were incubated for 1 hour at 37° C. Supernatants containing released calcein from complement lysed cells were transferred to fresh flat bottom microtiter plates. The remaining intact cells with retained Calcein AM were lysed using 0.05 mL 1% sodium dodecylsulfate (SDS). Released and retained fractions were read on a cytofluor 2350 spectrophotometer (Millipore, Bedford, Mass.) at 485 nm. Data is presented as percent cell death.

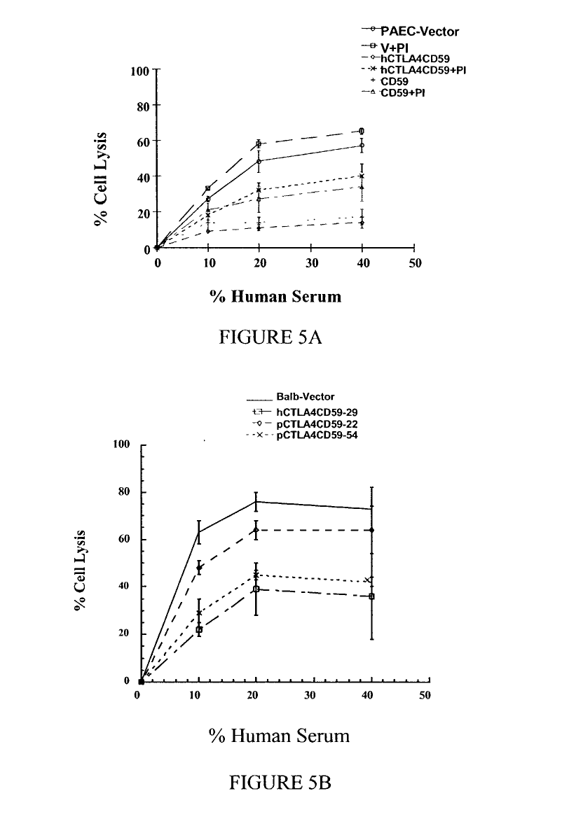

The hCTLA4-hCD59 PAECs were assayed in human serum complement-mediated cell lysis experiments to determine if the CD59 moiety was functional. FIG. 5A illustrates the percentage of cell death with increasing concentrations of human serum. The vector control PAEC susceptibility to human serum is dose dependent and results in approximately 60% cell death in 40% serum. A human CD59 PAEC cell line (Kennedy et al., 1994) served as a positive control and exhibited reduced sensitivity to human serum lysis, 17% cell lysis in 40% serum. The hCTLA4-hCD59 cells paralleled the hCD59 cell line resistance at all concentrations of serum tested (FIG. 5A). To further demonstrate that the CD59 moiety was conferring complement resistance, PI-PLC treated cells were used in the serum cytolysis assay. Phosphatidyl inositol phospholipase C (PI-PLC) enzymatically cleaves GPI-linked molecules from the surface of cells and therefore should cleave the hCTLA4-hCD59 and hCD59 molecules from the transduced PAECs. Loss of cell surface expression following PI-PLC treatment was assessed by flow cytometry. As expected, removing the hCTLA4-hCD59 molecule from the surface resulted in an increase in cell sensitivity to human serum (FIG. 5A). There was no significant effect on the vector control cells following PI-PLC treatment. However, both the hCD59 and the hCTLA4-hCD59 PAECs showed an increase in complement-mediated cell lysis following PI-PLC treatment. Both the hCD59 and the hCTLA4-hCD59 cells were 2-3 fold more sensitive to human serum. Taken together, these results indicate that the CD59 moiety is functioning to protect the hCTLA4-hCD59 PAECs from human serum cytolysis.

The Balb/3T3 transfected cell lines were also analyzed in human serum complement-mediated lysis assays to demonstrate that the human CD59 moiety in the porcine CTLA4 constructs was functionally active. FIG. 5B illustrates the level of serum complement resistance in each of the cell lines expressing the various CTLA4-hCD59 chimeras. The pBABE vector alone transfected cells were lysed by human serum at all concentrations tested. The pCTLA4(AS)₃-hCD59-22 clone provided some protection against complement-mediated cytolysis that was significant at 10% and 20% serum but failed to significantly protect at 40% serum as compared to the vector control cells (FIG. 5B). Both the pCTLA4(G)₆-hCD59-54 and hCTLA4-hCD59-29 Balb/3T3 transfectants demonstrated protection at all concentrations of serum as compared to the control cell population (FIG. 5B).

The CD59 complement regulatory activity is seen to be functional even with the CTLA4 moiety engaging CD80/CD86, although direct molecular interactions following complement deposition were not measured. The pCTLA4(AS)₃-hCD59-22 clone was less effective at inhibiting complement on the Balb/3T3 cell as compared to the hCTLA4-hCD59-clone 29 and pCTLA4(G)₆-hCD59-54 clone (FIG. 5B). The difference between clone 54 and clone 22 was the different amino acid linker separating the pCTLA4 and hCD59 domains. Conformational differences in the mature pCTLA4-hCD59 molecules may result in differential complement regulatory activity, where the (AS)₃ linker is confounding, however this molecule is expressed at approximately 10-fold higher levels and may compensate for the reduced activity (i.e., hCTLA4-hCD59-29 and pCTLA4(G)₆-hCD59-54 exhibit equal levels of survival, however the clone 54 has 10-fold less expression as determined by FACS (FIGS. 5B, 4D and 4F, respectively).

### EXAMPLE 12

### Costimulation Assay

The costimulatory capacity of the PAEC or 3T3 was assayed using a modified endothelial cell costimulation assay (Murray et al., 1994). Fifty thousand antigen presenting cells were seeded per well of a 96 well cluster (Becton Dickinson, Franklin Lakes, N.J.) 24 hours prior to co-culturing with Jurkat or T cells. Responding cells were added at a 2:1 ratio. The following reagents were added in complete medium to these final concentrations: 5 μg/mL mouse anti human CD28 (Pharmingen, San Diego, Calif.) or mouse anti porcine B7.2; 10 μg/mL mouse anti human CD 152 or rat anti mouse B7.1; 5 μg/well human CD152Ig or porcine CD 152. Thirty minutes later the lectin, phytohemagglutinin was added to a final concentration of 10 μg/mL and cultured overnight at 37° C. Cell free supernatants were collected 22 hours later and assayed for human or mouse IL2 by ELISA (R&D Systems, Minneapolis, Minn.). Jurkat supernatants were tested undiluted, while T cell supernatants were diluted 1:10. Plates were read on a microplate reader 3550 (Biorad, Hercules, Calif.) at 450 nm.

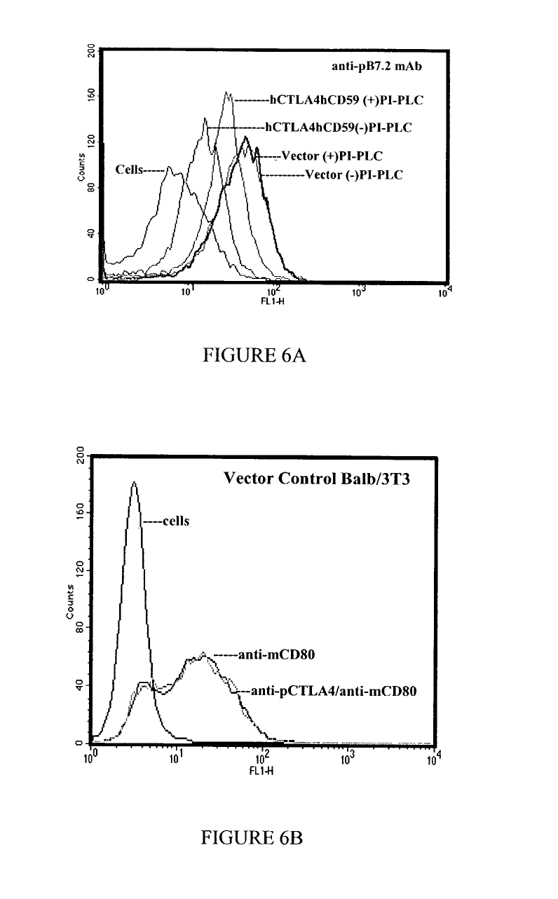

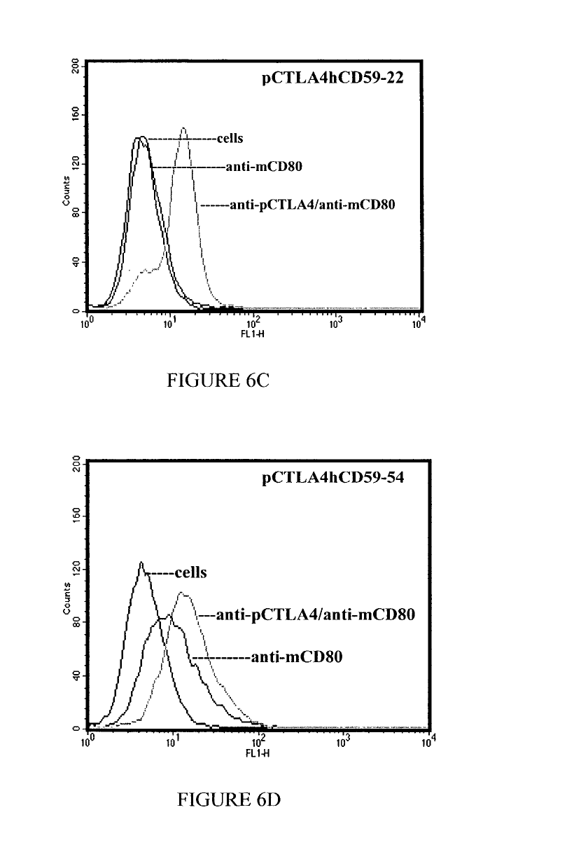

CTLA4 expressed on the surface of the hCTLA4-hCD59 expressing cells should theoretically bind CD80 and CD86 molecules thereby preventing CD28-CD80 and/or CD28-CD86 engagement and thus reduce activation of T cells. The possibility that the engineered CTLA4 could bind B7 in cis on the surface of the hCTLA4-hCD59 cells was investigated. FIG. 6A shows anti-B7.2 antibody reactivity on the vector control PAECs or hCTLA4-hCD59 PAECs, either treated or untreated with PI-PLC. Interestingly, when an anti-pCD86 monoclonal antibody was incubated with the hCTLA4-hCD59 PAECs, reduced reactivity was observed as compared to vector control cells (FIG. 6A). However, when the hCTLA4-hCD59 molecule was removed from the cell surface, an increase in anti-pCD86 antibody reactivity was observed. The result was approximately a two-fold shift in mean FL1, indicating that pCD86 was accessible to the anti-pCD86 antibody after the chimeric molecule was removed from the PAEC cell surface (FIG. 6A). Vector control cells show no change in the amount of anti-pCD86 antibody bound when treated with PI-PLC (FIG. 6A). The Balb/3T3 transfected cells expressed murine CD80 as determined by flow cytometry, however there was no evidence of CD86 expression. To assess cis binding of pCTLA4 to murine CD80 in the context of the chimeric molecules, antibody blocking experiments were utilized (FIGS. 6B-D). The transfected cells were treated with the anti-pCTLA4 polyclonal antibody to inhibit the pCTLA4 moiety from binding to CD80 on the mouse cell surface. The cells were subsequently incubated with an anti-mCD80 rat monoclonal antibody to detect mCD80. The vector control Balb/3T3 transfected cells showed equal reactivity with the anti-mCD80 monoclonal antibody regardless of ALP-67 (i.e., anti-pCTLA4) treatment (FIG. 6B). However both the pCTLA4(AS)₃-hCD59-22 and pCTLA4(G)₆-hCD59-54 exhibited an increase in anti-mCD80 antibody reactivity following the pre-treatment with ALP-67, indicating that the chimeric molecules were inhibiting anti-mCD80 access to the CD80 antigen (FIGS. 6C-D).

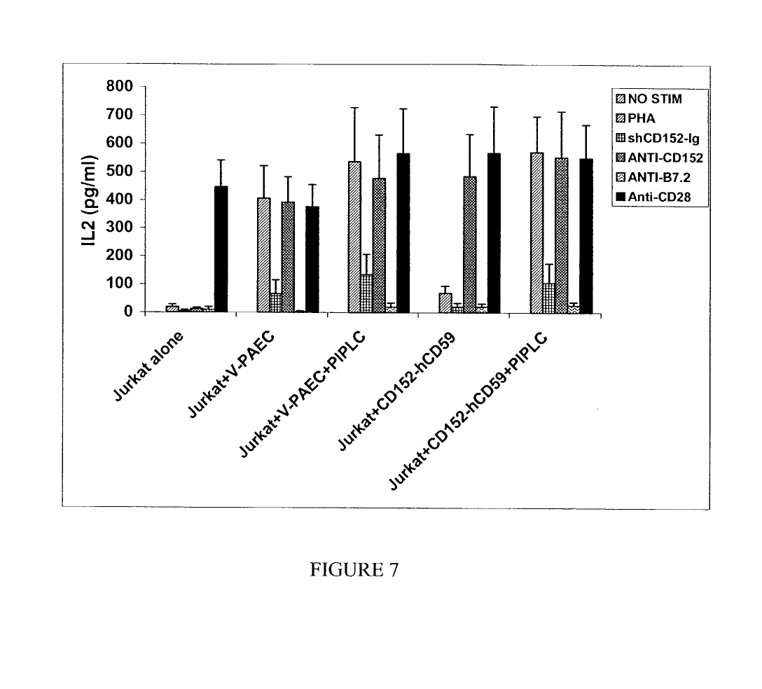

To test the function of the chimeric molecules at inhibiting T cell activation, costimulation assays were performed using the human T cell line Jurkat, which cells are known to produce IL-2 following costimulation through the CD28 pathway (Thompson et al., 1989; Shapiro et al., 1998). The amount of IL-2 elicited from Jurkat cells in the presence or absence of pig aortic endothelial cells as antigen presenting cells depends upon primary and secondary stimulatory signals. Without the secondary co-stimulatory signal provided by an APC or anti CD28, Jurkat cells remain unactivated and secrete little to no IL2. The assay utilizes the lectin phytohemagglutinin (PHA) to crosslink the T-cell receptor complex and stimulate the primary signal. Initially, the hCTLA4-hCD59 PAECs were tested for their ability to elicit an IL-2 response from Jurkat cells. FIG. 7 depicts the amount of IL-2 elicited from Jurkat cells in the absence or presence of APCs which were either untreated or PI-PLC treated. Jurkat cells in the absence of APC costimulation can be induced to secrete IL-2 by crosslinking the T cell receptor with PHA followed by crosslinking CD28 with an anti-CD28 antibody. This treatment resulted in 446 pg/mL of IL-2 (FIG. 7). In the absence of PHA, vector control PAECs cannot elicit an IL-2 response from Jurkat cells (FIG. 7). Following PHA/vector-PAEC co-culture, the Jurkat cells produced approximately 400 pg/mL of IL-2. This IL-2 response can be prevented in the presence of a blocking antibody to pig CD86 or with sCTLA4-Ig. Anti-hCTLA4, which is known to block the binding of CTLA4 to CD80 and CD86, has no effect on IL-2 production when vector control cells are used as APC (FIG. 7). Jurkat cells co-cultured with the vector control PAECs following PI-PLC treatment had a slight positive effect on the costimulation observed as an increase in IL-2 synthesis as compared to the untreated vector control cells. However, when hCTLA4-hCD59 cells were used as APCs, very little IL-2 was secreted from Jurkat responder cells. An approximately 5 fold decrease in IL-2 production was observed (FIG. 7). The level of IL-2 secretion was further reduced following treatment with the anti-pCD86 antibody or sCTLA4-Ig (FIG. 7). Specific blocking by treatment with antiCTLA4 (anti-CD152) restored costimulation, which resulted in secretion of IL-2 to 484 pg/mL (FIG. 7). The anti-CD28 antibody treatment stimulated the Jurkat cells directly and served as an internal control. PI-PLC treatment of the hCTLA4-hCD59 PAECs resulted in loss of costimulation blockade due to the loss of the hCTLA4-hCD59 molecule, which led to Jurkat activation and IL-2 secretion (570 pg/mL, FIG. 7). Costimulation could still be inhibited with anti-pCD86 and sCTLA4-Ig. No effect was seen with the anti-hCTLA4 antibody. Taken together, the anti-CTLA4 blocking antibody result and the PI-PLC experiment indicated that the reduction in IL-2 elicited from the hCTLA4-hCD59 PAEC was specifically due to the presence of the CTLA4 molecule on the engineered APC.

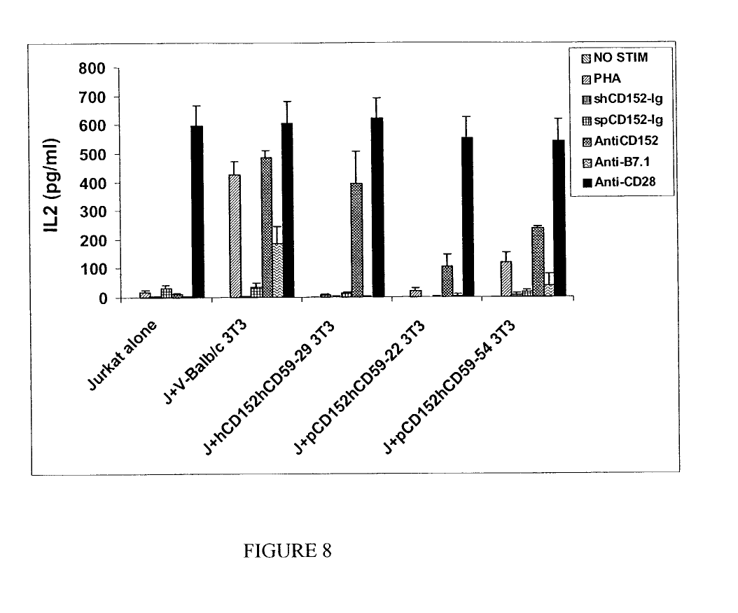

The transfected Balb/3T3 cells were tested in Jurkat co-culture assays to determine if the human and porcine chimeric molecules could inhibit costimulation through mCD80-CD28 ligation. The vector control cells stimulated Jurkat cells to secrete approximately 600 pg/mL of IL-2 following PHA treatment (FIG. 8). This response could be blocked with human sCTLA4-Ig or porcine sCTLA4-Ig (FIG. 8). Blocking experiments with the anti-mCD80 monoclonal antibody only resulted in a two-fold decrease in IL-2 secretion. The anti-CTLA4 and anti-CD28 treatments served as controls as described above. In all of the chimeric molecule transfectants a significant reduction in IL-2 was observed. The hCTLA4-hCD59-29 clone completely inhibited costimulation that could be restored with anti-hCTLA4 treatment (FIG. 8). The porcine chimeric molecule, pCTLA4(AS)₃-hCD59-22 also completely inhibited IL-2 synthesis. Treatment with the anti-pCTLA4 polyclonal only partially restored costimulation (FIG. 8). The porcine chimeric molecule, pCTLA4(G)₆-hCD59-54 significantly inhibited IL-2 secretion, but was less effective than clone 22 or the human clone 29. Similar to clone 22, treatment with the anti-pCTLA4 polyclonal only partially restored costimulation (FIG. 8).

In the process of cloning the pCTLA4 and generating the chimeric pCTLA4-hCD59 molecules, a soluble pCTLA4-Ig molecule was also constructed. The soluble pCTLA4-Ig protein was purified from human 293 transfection supernatants and tested in the Jurkat costimulation assays. Porcine spCTLA4-Ig inhibited costimulation and was equally effective on mCD80-hCD28 stimulation as the human control (FIG. 8).

The above results demonstrate that co-stimulatory blockade is seen for all three versions of the chimeras (FIGS. 7 and 8). PI-PLC and antibody blocking experiments determined that human or porcine CTLA4 domains bound to pig CD86 and mouse CD80 in cis. Determining that the chimeric molecules exhibit cis regulatory activity that inhibits co-stimulation is a novel feature of the present engineered molecules.

### EXAMPLE 13

### Flow Cytometric Analysis of Porcine Cells for NK Cell Experiments

Direct fluorescence of cell-surface carbohydrate epitopes was performed with fluorescein isothiocyanate (FITC)-conjugated lectins: IB4 lectin isolated from Griffonia simplicifolia (EY Laboratories, Inc. San Mateo, Calif.) that detects Galα-1,3-Gal (Hayes and Goldstein, 1974) and UEAI lectin isolated from Ulex europaeus (EY Laboratories) that detects H-substance (Matsumoto and Osawa, 1969). Indirect immunofluorescence of hCD59 and hCTLA4 was performed with the specific mouse monoclonal antibodies BRA10G (BIODESIGN International, Kennebunk, Me.) and BNI3 (Immunotech, Marseille, France), respectively. SLA class I was detected with the murine monoclonal antibody PT85A (VMRD, Inc., Pullman, Wash.). Porcine CD86 expression was detected with 4F9.86 and 5B9.88 mAbs, whereas pCD80 expression was assessed with the rabbit polyclonal antibody ALP61 (Faas et al., 2000). These last three antibodies have been developed by Alexion Pharmaceuticals (Cheshire, Conn.). Goat anti-rabbit IgG and goat anti-mouse IgG, IgA and IgM (Zymed Laboratories, Inc., So. San Francisco, Calif.) FITC-conjugated antisera were used to detect specific antibody binding. Cell surface expression was then measured by flow cytometry on a Becton Dickinson FACSort.

### EXAMPLE 14

### NK Cell Isolation and Culture

The human NK cell line NK92 (obtained from H. G. Klingemann, Rush University, Chicago, Ill.) was first described by Gong et al. (1994). These cells were cultured in Myelocult H5100 media (StemCell Technologies, Vancouver, B.C.) supplemented with 100 U/mL of human recombinant IL-2 (Hoffman-La Roche Inc., Nutley, N.J.). The human YTS cells (obtained from G. Cohen, Massachusetts General Hospital, Boston), a subline of the YT NK leukemia cell line (Yoneda et al., 1992), were cultured in Iscove's modified medium supplemented with 10% FCS and L-glutamine. Human NK cells were purified from PBL from different donors as previously described (Watzl et al., 2000). First, PBL were isolated from buffy coats by centrifugation with Lymphocyte Separation Medium (ICN Biomedicals Inc., Aurora, Ohio). NK cells were subsequently enriched by depletion of other cell types utilizing the magnetic-activated cell sorter NK cell Isolation Kit (Miltenyi Biotech, Auburn, Calif.). Freshly isolated NK populations were 90-99% CD3⁻ and CD56⁺ by flow cytometric analysis. The Iscove's modified medium plus 10% human serum, supplemented with rIL-2 (100 U/mL) and 10% purified human IL-2 (Hemagen, Columbia, Md.), was used for culturing and expanding NK cells.

### EXAMPLE 15

### Flow Cytometric Analysis of NK Cells

For surface staining, cells were incubated with the following antibodies: anti-CD3 (UCHT1, -R-PE from Pharmingen, San Diego, Calif.), anti-CD56 (B159, -R-PE from Pharmingen), anti-CD28 (CD28.2, -FITC from Pharmingen) and anti-CD28 variant (YTH913.12, -FITC from Serotec, Oxford, UK). Directly conjugated isotype matched antibodies (Pharmingen) were used as negative controls.

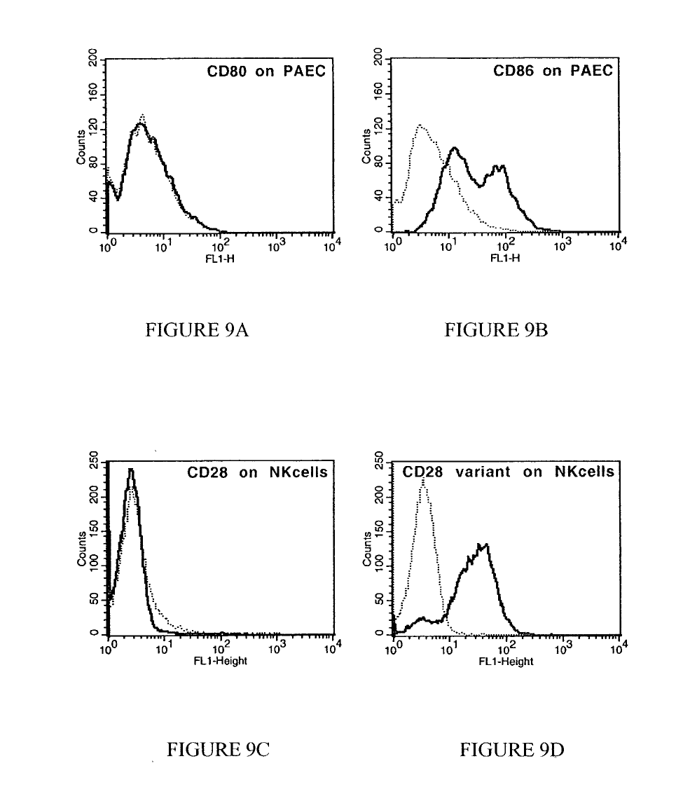

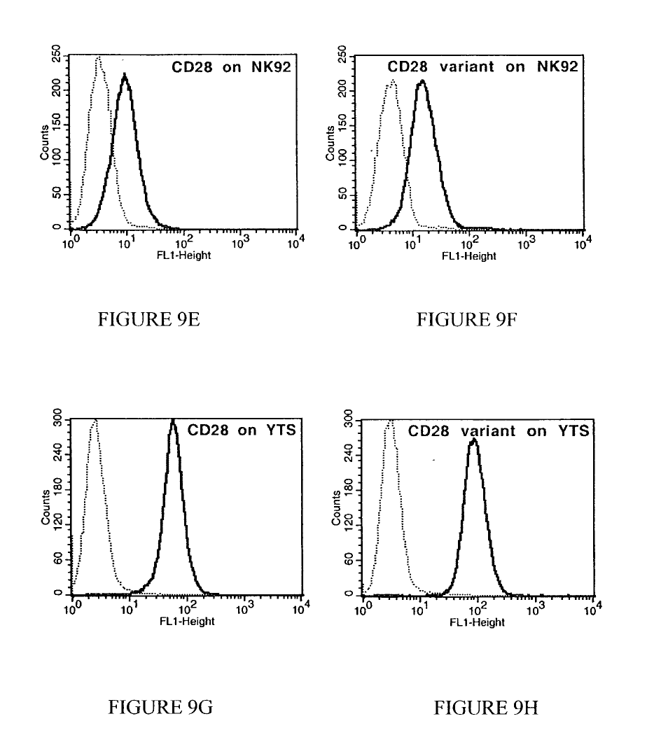

The levels of CD80 and CD86 expression in the primary cultured PAEC were assessed by flow cytometric analysis (FIGS. 9A-B). To this end, a polyclonal antibody (ALP61) specific for porcine CD80 (Faas et al., 2000) was utilized (FIG. 9A) and several monoclonal antibodies specific for porcine CD86 were utilized. Data generated with 4F9.86, an anti-CD86 antibody, are shown in FIG. 9B. These experiments confirmed previous observations that PAEC express CD86 (Maher et al., 1996). However, in the standard culture conditions, CD80 cell-surface expression was not detected (FIG. 9A). These results indicate that in resting conditions PAEC express CD86, but not CD80.

### EXAMPLE 16

### NK Cell-Mediated Cytotoxicity Assays

Cytotoxicity assays were performed as previously described (Artrip et al., 1999). Briefly, target cells (PAEC or porcine fibroblasts) were seeded and grown overnight to confluence in 96-well plates. The next day, cells were washed in Hank's solution (Biosource International, Camarillo, Calif.), labeled with ⁵¹Cr (2-4 μCi/well, Amersham Pharmacia Biotech, Piscataway, N.J.) for 60 minutes and washed extensively again before the assay. The assay was conducted for 4 hours after addition of the effector cells (NK92, YTS or IL-2-activated NK cells) in the indicated effector:target ratios in a 200 μL final volume. Data are presented as percentage of specific lysis and calculated as previously described (Artrip et al., 1999). To block porcine CD86, target cells were incubated for 20 minutes at room temperature with the specific mAb 5B9.88 and washed prior to the addition of the NK92 effector cells.

To assess the susceptibility of porcine cells to NK cell-mediated cytotoxicity, freshly isolated NK cells, as well as two tumor-derived NK cell lines, NK92 and YTS, were utilized. The NK92 and YTS cells were chosen because they express CD28 (Gong et al., 1994; Azuma et al., 1992). Whereas expression of CD28 has not been observed in human NK cells (Wilson et al., 1999), Galea-Lauri et al. (1999) have demonstrated expression of a CD28 variant in some NK cells that is detected with the monoclonal antibody YTH913.12. This molecule is here denoted “CD28 variant”. To determine the molecules involved in this system, the expression of CD28 and CD28 variant was evaluated in all NK cell populations assayed, as well as in NK92 and YTS cell lines (FIGS. 9C-H). Reactivity towards CD28 variant was consistently observed in all preparations of IL-2 activated NK cells isolated from different donors (FIG. 9D), while no expression of CD28 was detected on these cells (FIG. 9C). The cell preparations contained from 60 to 90% of CD28 variant positive cells. The NK92 and YTS cell lines showed expression of both CD28 (FIGS. 9E and 9G) and CD28 variant (FIGS. 9F and 9H), with YTS exhibiting the highest levels of expression for both molecules (FIGS. 9G-H).

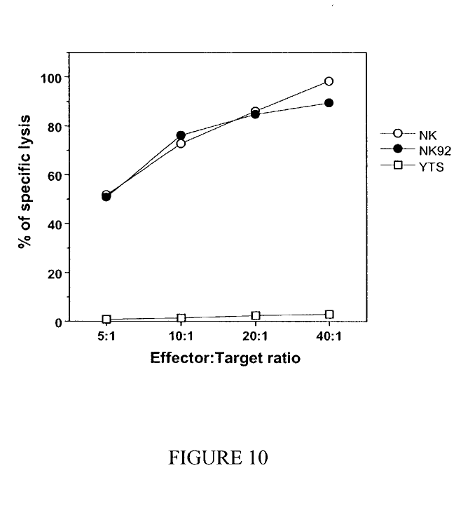

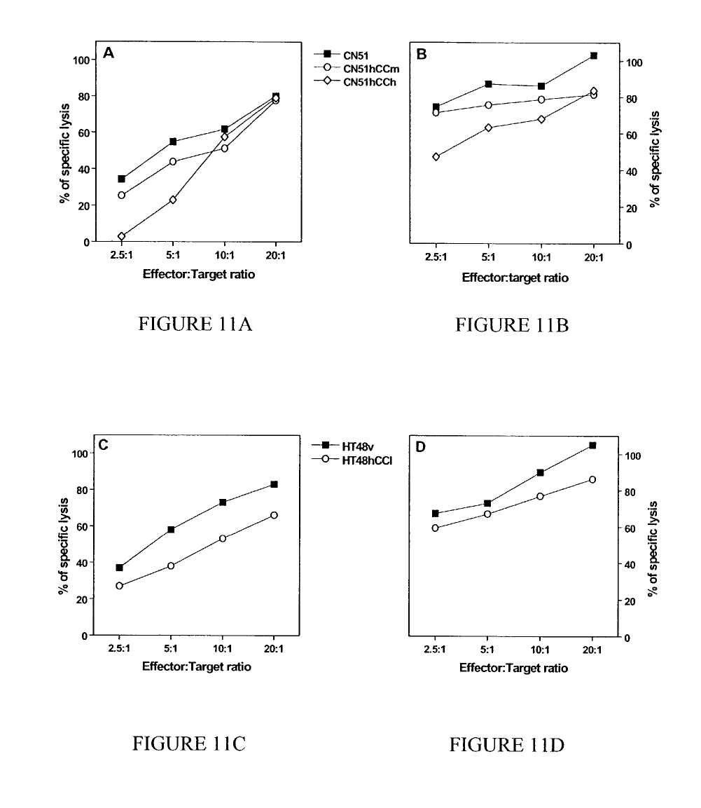

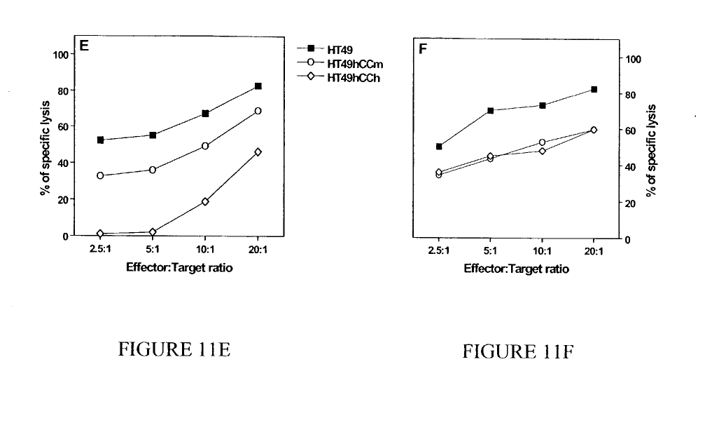

NK cell-mediated cytotoxicity towards porcine cells was tested using IL-2 activated NK cells, NK92 and YTS cells in the absence of human serum (FIG. 10). Porcine cells were highly susceptible to lysis mediated by NK92 and activated NK cells in accordance with previous observations (Goodman et al., 1997; Donnelly et al., 1997; Itescu et al., 1996). Interestingly, YTS showed no cytotoxicity towards PAEC (FIG. 10). Similar results were obtained when these NK cell preparations were tested against HT-transgenic PAEC transduced with pBABE vector alone (FIGS. 11C-D and data not shown). This protection from YTS-mediated lysis may be explained, at least in part, by the absence of CD80 on PAEC. Recent studies indicate that YTS triggering is mediated through CD80 and not CD86 (Montel et al., 1995; Luque et al., 2000).

### EXAMPLE 17

### Expression of hCTLA4-hCD59 Chimeric Molecule on PAEC Reduces CD86 Accessibility and NK Cell-Mediated Lysis

The hCTLA4-hCD59 chimeric molecule was designed to block CD86 on the porcine cell. This chimeric molecule binds to CD86 in cis and inhibits costimulation provided by PAEC to human T cells. To study whether porcine CD86 contributes to human NK cell triggering and to assess the effect of CD86 blockade, hCTLA4-hCD59 was expressed in primary cultured PAEC from 3 different pigs. PAEC from the non-transgenic control #51 and 2 HT-transgenic pigs, #48 and #49, were transduced with pCTLA4hCD59BABEpuro. Several clones from each cell line were generated and selected based upon various levels of hCTLA4-hCD59 (hCC) expression. A low expressing clone, hCCl, a medium level expressor, hCCm, and a high level expressing clone hCCh, were identified. The phenotypic characterization by flow cytometry of these selected clones and their controls is summarized in Table I. Expression of H-epitope and the accompanying reduction of Galα-1,3-Gal antigen was confirmed in the HT-transgenic cells by staining with UEAI and IB4 lectins, respectively (Table I). Both BRA10G, specific for hCD59, and BNI3, specific for hCTLA4, detected comparable hCTLA4-hCD59 expression levels in the transduced clones. Most importantly, expression of hCTLA4-hCD59 correlated inversely with antibody reactivity towards CD86, as detected with the monoclonal antibody 4F9.86 (Table I). CD86 was inaccessible to 4F9.86 in the high hCTLA4-hCD59-expressing clones (Table I), confirming the efficacy of this approach. The level of SLA class I expression was also assessed by staining with PT85A (Table I), given that SLA I may be involved in signaling to NK cells. Moreover, this parameter is a good indicator of the overall gene expression levels in the primary cultured cells. A trend towards lower levels of SLA I expression in the transduced clones relative to non-transduced cells was observed, but these differences were not significant. Experiments were conducted that showed no effect of this variation, or the presence or absence of pBABE vector alone, in the susceptibility of these cells to NK-cell mediated lysis.

The CD86 contribution to NK cell triggering was assessed with a series of NK cell-mediated cytotoxicity assays in which the different clones of PAEC were co-cultured with the CD16-deficient NK92 cells or IL-2 activated human NK cells (FIGS. 11A-F). All assays were carried out in the absence of human serum. Expression of hCTLA4-hCD59 led to some level of

TABLE I
Flow Cytometric Analysis of Control and
HT-transgenic PAEC Transduced with hCTLA4-hCD59^{a}

| | | | | | |
|---|---|---|---|---|---|
| UEA-I^{c} | GS-IB4^{c} | BRA10G^{b,d} | BNI3^{b,d} | 4F9.86^{b,d} | PT85A^{d} |

| | | | | | | | |
|---|---|---|---|---|---|---|---|
| Control 51 | 2.7 ± 0.6 | 289.8 ± 63.5 | 0.4 ± 0.2 | 0.9 ± 0.35 | 27.2 ± 4.9 | 76.9 ± 22.1 | |
| Control-hCCm | 1.7 ± 0.4 | 211.4 ± 59.6 | 85.6 ± 7.5** | 101.2 ± 12.3** | 9.3 ± 0.3* | 56.5 ± 7.9 | |
| Control-hCCh | 2 ± 0.8 | 236.6 ± 84.5 | 110.9 ± 21.3** | 111.8 ± 16.7** | 3.8 ± 0.5* | 40.4 ± 8 | |
| HT 48-vector | 351.6 ± 91.9 | 124.6 ± 31.6 | 0.03 ± 0.01 | 0.08 ± 0.08 | 35.7 ± 4.8 | 43.1 ± 2.2 | |
| HT 48-hCCl | 369.2 ± 35.3 | 64 ± 17.4 | 10.4 ± 2.2** | 10.2 ± 1.4** | 16 ± 1.9* | 47.3 ± 2.9 | |
| HT 49 | 705.6 ± 397 | 190.6 ± 112.5 | 0.01 ± 0.01 | 0.3 ± 0.1 | 35.8 ± 12 | 113.4 ± 45.5 | |
| HT 49-hCCm | 288.2 ± 92 | 29.8 ± 10.7 | 31.7 ± 7.9* | 30.9 ± 10.8* | 10 ± 2.4* | 39 ± 12.4 | |
| HT 49-hCCh | 464.4 ± 162 | 63.1 ± 18.3 | 76.2 ± 7** | | 82.6 ± 14.2** | 3.5 ± 0.3* | 34 ± 9.9 |

^{a}All values are expressed as the mean ± SE of the mean fluorescence intensity (n=4).
The specificities of the lectins used are as follows: UEAI/H-epitope, GS-IB4/Galα1, 3 Gal epitope.
The specificities of the antibodies used are as follows: BRA10G/hCD59, BNI3/hCD 152, 4F9.86/pCD86, PT85A/SLAI.
^{b}Significant differences were detected between hCC-transduced and their corresponding control cells, *p ≦ 0.05, **p ≦ 0.005.
^{c}Significant differences were detected between HT-transgenic and control cells, p ≦ 0.05.
^{d}No differences were observed when comparing HT-transgenic and control cells.

protection from cytotoxicity mediated by both NK92 and activated NK cells (FIGS. 11A-F). Blockade of porcine CD86 was more effective in reducing NK92 (FIGS. 11A, 11C and 11E) than NK cell-mediated lysis (FIGS. 11B, 11D and 11F). The reduction in lysis was more dramatic in the highest hCTLA4-hCD59-expressing clones, indicating a role of CD86 in triggering NK-cell mediated cytotoxicity of porcine cells.

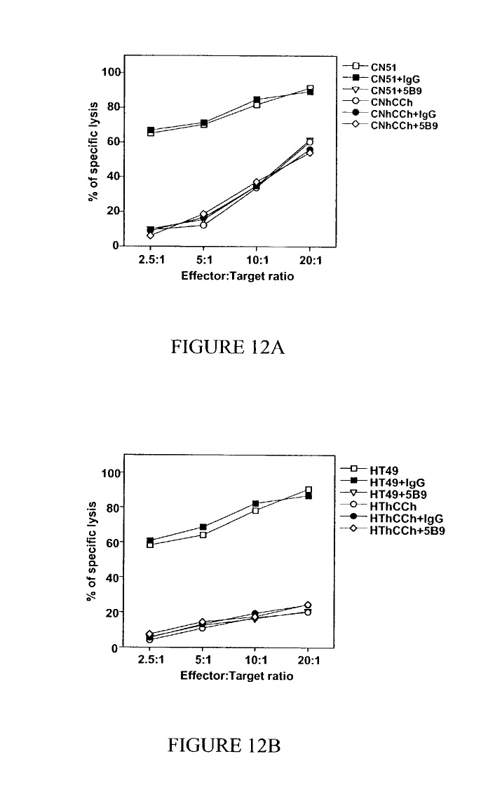

The specific anti-pCD86 blocking antibody 5B9.88 was utilized in NK92-mediated cytotoxicity assays of PAEC to confirm the foregoing observations (FIGS. 12A-B). This antibody has shown very similar reactivity by flow cytometry to all the PAEC lines assayed when compared to 4B9.86 antibody and has shown blocking activity in a human anti-porcine mixed lymphocyte reaction (MLR) (A. Mickle, personal communication). The highest hCTLA4-hCD59-expressing clones were also included in the assays to reconfirm that the reduction in CD86 accessibility was responsible for the observed protective effect. Whereas the addition of IgG1 isotype control did not affect the NK92-mediated lysis of control #51 and HT-transgenic #49 cells, incubation with 5B9.88 led to a reduction in lysis identical to that achieved by high expression of hCC (FIGS. 12A-B). Moreover, addition of either isotype control or 5B9.88 antibodies did not alter the susceptibility to lysis of the high hCC-expressing PAEC (FIGS. 12A-B). These data demonstrate that ligation to CD86 on porcine cells contributes to human NK cell triggering and that blockade of porcine CD86 confers partial protection from NK cell-mediated cytotoxicity. It is of interest to note that this triggering pathway differs from that described in the recognition of PAEC by human T cells via CD28 (Murray et al., 1994). In this case, the CD28 variant detected in NK cells is probably the only one involved.

### EXAMPLE 18

### Effect of Carbohydrate Remodeling on NK92 and NK Cell-Mediated Cytotoxicity

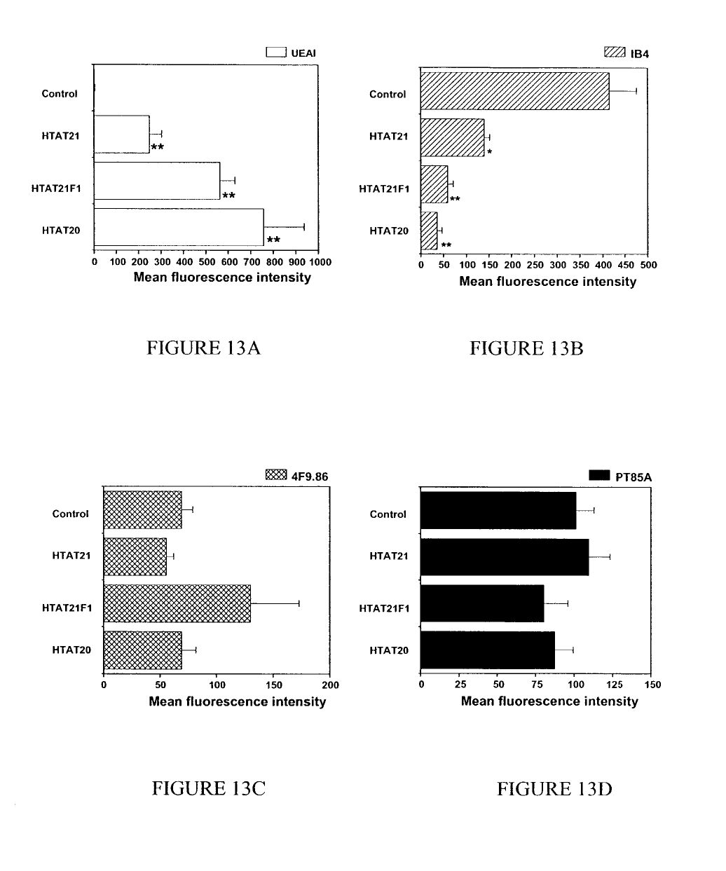

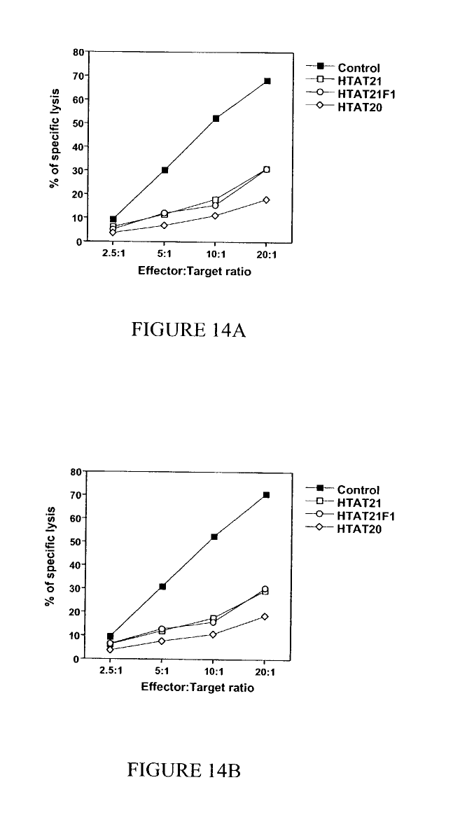

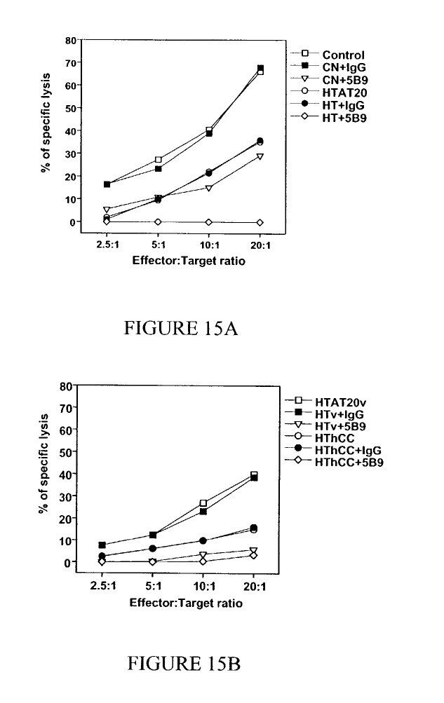

CD86 blockade reduces, but does not completely abrogate NK cell-mediated cytotoxicity (FIGS. 11A-F). In accordance with previous studies (Artrip et al., 1999; Sheikh et al., 2000), there are probably multiple molecules controlling this process. Galα-1,3-Gal and other carbohydrate antigens may also trigger signals that render PAEC more susceptible to human NK cell-mediated lysis than HUVEC. In this regard, it was observed that the 2 HT-transgenic PAEC assayed showed a trend to be more resistant to lysis, especially the clones expressing high levels of hCTLA4-hCD59 (FIGS. 11A-F and 12A-B). This trend was also observed when the control #51, and the HT #48-vector and #49 PAEC were simultaneously assayed with NK92 cells. Due to availability, the HT-transgenic PAEC which were utilized were derived from pigs from the moderate expressing transgenic line HTAT21 (Costa et al., 1999; Costa et al., 2002). As a marked reduction in Galα-1,3-Gal antigen in fibroblasts isolated from pigs of the high expressing HTAT20 line (Costa et al., 1999) was observed, these cells were then assayed in an NK cell-mediated cytotoxicity assay. Fibroblasts isolated from the two founder pigs AT20 and AT21, from F₁ descendents of each line, as well as a control pig, were included in these experiments. These cells could be distributed in 4 groups that differed in their expression levels of H- and Galα-1,3-Gal epitopes. Representative results from one single cell line per group are shown (FIGS. 13A-D and 14A-B). Significant differences in lectin reactivity were detected between the HT-transgenic and control cells. Non-transgenic cells had the highest cell surface expression of Galα-1,3-Gal epitope (FIG. 13B). In inverse correlation to H-epitope expression (FIG. 13A), relatively high levels of Galα-1,3-Gal epitope remained on HTAT21-transgenic fibroblasts (mosaic founder), HTAT21F₁ cells expressed moderate levels and HTAT20-transgenic fibroblasts had the lowest Galα-1,3-Gal expression. No significant differences were detected between these cells in CD86 (FIG. 13C) and SLA I expression (FIG. 13D). Expression of porcine CD80 was negligible on the fibroblasts. The efficacy of HT expression in providing protection from NK cell-mediated cytotoxicity was confirmed when the transgenic fibroblasts were challenged with NK92 (FIG. 14A) and IL-2 activated NK cells (FIG. 14B) in the absence of human serum. In all assays, control cells were the most susceptible to lysis, whereas cells from the HTAT20 line were the most resistant (FIGS. 14A-B). Cells from the HTAT21 line were intermediate in resistance (FIGS. 14A-B). This resistance is most likely due to the reduction in Galα-1,3-Gal epitope expression on the transgenic cells (Artrip et al., 1999).

### EXAMPLE 19

### Co-Expression of High Levels of hCTLA4-hCD59 and H-Transferase Confers Protection from NK-Cell Mediated Lysis

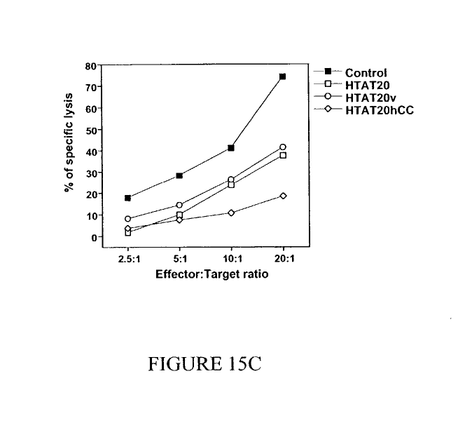

To finally assess whether the combination of high HT expression and CD86 blockade confers maximal protection from NK cell-mediated lysis, HTAT20 fibroblasts were transduced with hCTLA4hCD59BABEpuro. HTAT20 cells transduced with the vector alone were also generated for controls. The pool of HTAT20-hCC transduced cells showed good expression of hCTLA4-hCD59 and a 60% reduction in anti-CD86 4F9.86 antibody reactivity when compared to HTAT20-vector alone (Mean fluorescence intensity 40.6±1.6 versus 98.9±1.5, respectively) as determined by flow cytometric analysis. To further study the effect of complete CD86 blockade, the blocking antibody 5B9.88 was also used in the cytotoxicity assays with NK92 cells (FIGS. 15A-B). Whereas the addition of 5B9.88 to control porcine fibroblasts provided partial protection from NK92-mediated lysis, this antibody completely abrogated NK92-mediated cytotoxicity towards HTAT20 transgenic cells (FIG. 15A). Complete resistance was also achieved when HTAT20 vector- or hCC-transduced cells were treated with the CD86 blocking antibody (FIG. 15B). Moreover, HTAT20 hCC-expressing cells showed intermediate resistance, correlating with the reduction in CD86 accessibility (FIG. 15B). This level of protection was reproduced when the HTAT20-hCC fibroblasts were challenged with different IL-2 activated NK cell preparations (FIG. 15C), indicating that this combinatorial approach confers resistance from human NK cell-mediated cytotoxicity.

### EXAMPLE 20

### Statistical Analysis

The indicated values are expressed as the means±SE. Statistical analysis was carried out using the Student-Newmann-Keuls test. Differences were considered statistically significant at p≦0.05.

It will be understood that various modifications may be made to the embodiments disclosed herein. For example, the C5b-9 inhibitory domain and/or the T-cell or NK cell inhibitory domain may be modified by creating amino acid substitutions or nucleic acid mutations, provided at least some complement regulatory activity and some T-cell or NK cell inhibitory activity remains after such modifications. Similarly, the nucleotide sequences of the chimeric protein may be modified by creating nucleic acid mutations which do not significantly change the encoded amino acid sequences, including third nucleotide changes in degenerate codons (and other “silent” mutations that do not change the encoded amino acid sequence). Mutations which result in conservative or silent amino acid substitution for an encoded amino acid while leaving the characteristics of the chimeric proteins essentially unchanged are also within the scope of this disclosure. As used herein, a conservative substitution is one in which an amino acid is substituted for another amino acid that has similar properties, such that one skilled in the art of peptide chemistry would expect the secondary structure and hydropathic nature of the polypeptide to be substantially unchanged. As a nonlimiting example, the following groups of amino acids represent conservative changes: (1) ala, pro, gly, glu, asp, gln, asn, ser, thr; (2) cys, ser, tyr, thr; (3) val, ile, leu, met, ala, phe; (4) lys, arg, his; and (5) phe, tyr, trp, his. Substitutions may also include nonnaturally occurring amino acids. A conservative substitution may also, or alternatively, contain other modifications, including the deletion or addition of amino acids that have minimal influence on the antigenic properties, secondary structure and hydropathic nature of the polypeptide. For example, a polypeptide may be conjugated to a signal (or leader) sequence at the N-terminal end of the protein that co-translationally or post-translationally directs transfer of the protein. The polypeptide may also be conjugated to a linker or other sequence for ease of synthesis, purification or identification of the polypeptide (e.g., poly-His), or to enhance binding of the polypeptide to a solid support. For example, a polypeptide may be conjugated to an immunoglobulin Fc region. Also included are sequences comprising changes that are found as naturally occurring allelic variants of the genes for the T-cell or NK cell inhibitory molecules and the C5b-9 or DAF inhibitory molecules used to create chimeric molecules described herein. All of the foregoing shall be considered as equivalents of the specific embodiments set forth herein. Therefore, the above description should not be construed as limiting, but merely as exemplifications of preferred embodiments. Those skilled in the art will envision other modifications within the scope of the claims appended hereto.

### LIST OF REFERENCES

The following references are incorporated herein by reference to more fully describe the state of the art. All patent and literature references cited in the present specification are hereby incorporated by reference in their entirety.

Adams E M, et al. (1991). J. Immunol. 147:3005-3011.

Artrip J H, et al. (1999). J. Biol. Chem. 274:10717-10722.

Aruffo A and Seed B (1987). Proc. Natl. Acad. Sci. USA 84:8573-8577.

Auchincloss H (1988). Transplantation 46:1-20.

Auchincloss H and Sachs D H (1998). Annu. Rev. Immunol. 16:433-470.

Ausubel F M, et al. (eds.) (1991). Current Protocols in Molecular Biology.

Azuma M, et al. (1992). J. Immunol. 149:1115-1123.

Blakely M L, et al. (1994). Transplantation 58:1059-1066.

Blazar B R, et al. (1995). Blood 85:2607-2618.

Bondioli K, et al. (2001). Mol. Reprod. Dev. 60:189-195.

Bradley, in Robertson (ed.) (1987). Teratocarcinomas and Embryonic Stem Cells a Practical Approach, IRL Press, Eynsham, Oxford, England.

Brinster R L, et al. (1985). Proc. Natl. Acad. Sci. USA 82:4438-4442.

Brunet J F, et al. (1987). Nature 328:267-270.

Brunet J F, et al. (1988). Immunol. Rev. 103:21-36.

Byrne G W, et al. (1997). Transplantation 63:149-155.

Candinas D, et al. (1996). Transplantation 62:1920-1927.

Carbone E, et al. (1997). J. Exp. Med. 185:2053-2060.

Chahine A A, et al. (1995). Transplantation 59:1313-1318.

Chavin K D, et al. (1992). Transplantation 54:286-291.

Coligan J E, et al. (eds.) (1992). Current Protocols in Immunology (J. Wiley and Sons (New York)).

Cosset F L, et al. (1990). J. Virol. 64:1070-1078.

Costa C, et al. (1999). FASEB J. 13:1762-1773.

Costa C, et al. (2002). Xenotransplantation 9:45.

Cozzi E, et al. (1997). In Xenotransplantation (Cooper D K C and Kemp E (eds.), Springer-Verlag, Berlin, Heidelberg), pp. 665-682.

Dalmasso A P, et al. (1992). Am. J. Pathol. 140:1157-1166.

Dariavach P, et al. (1988). Eur. J. Immunol. 18:1901-1905.

Davis E A, et al. (1996). Transplantation 62:1018-1023.

Diamond L E, et al. (1996). Transplantation 61:1241-1249.

Donnelly C E, et al. (1997). Cell. Immunol. 175:171-178.

Edge A S B, et al. (1998). Cell Transplant. 7: 525-539.

Eglitis M A and Anderson W F (1988). Biotechniques 6:608-614.

Evans M J, et al. (1995). J. Immunol. Methods 184:123-138.

Faas S J, et al. (2000). J. Immunol. 164:6340-6348.

Fearon D T (1979). Proc. Natl. Acad. Sci. USA 76:5867-5871.

Fodor W L, et al. (1994). Proc. Natl. Acad. Sci. USA 91:11153-11157.

Fodor W L, et al. (1995). J. Immunol. 155:4135-4138.

Galea-Lauri J, et al. (1999). J. Immunol. 163:62-70.

Goeddel D V (1990). Methods Enzymol. 185:3-7.

Gong J H, et al. (1994). Leukemia 8:652-658.

Goodman D J, et al. (1997). In Xenotransplantation (Cooper D K C and Kemp E, eds., Springer-Verlag, Berlin, Heidelberg), p. 77.

Harding F A, et al. (1992). Nature 356:607-609.

Hathcock K S, et al. (1994). J. Exp. Med. 180:631-640.

Hayes C E and Goldstein I J (1974). J. Biol. Chem. 249:1904-1914.

Hogan et al., in Manipulating the Mouse Embryo: A Laboratory Manual, Cold Spring Harbor Laboratory, Cold Spring Harbor, N.Y. (1986).

Inverardi L, et al. (1992). J. Immunol. 149:1416-1423.

Itescu S, et al. (1996). Transplantation 62:1927-1933.

Itescu S, et al. (1998). Hum. Immunol. 59:275-286.

Kawauchi M, et al. (1993). J. Thorac. Cardiovasc. Surg. 106:779-786.

Kennedy S P, et al. (1994). Transplantation 57:1494-1501.

Kobayashi T S, et al. (1997). Transplantation. 64:1255-1261.

Koike C, et al. (1996). Xenotransplantation 3:81-86.

Korman A J, et al. (1987). Proc. Natl. Acad. Sci. USA 84:2150-2154.

Koulmanda M, et al. (1995). Xenotransplantation 2:295.

Kroshus T J, et al. (1996). Transplantation 61:1513-1521.

Kumagai-Braesch M, et al. (1998). Xenotransplantation 5:132-145.

Lambrigts D, et al. (1998). Transplantation 66:547-561.

Larsen C P, et al. (1996). Nature 381:434-438.

Lenschow D J, et al. (1992). Science 257:789-792.

Leventhal J R, et al. (1993a). Transplantation 55:857-866.

Leventhal J R, et al. (1993b). Transplantation 56:1-8.

Leventhal J R, et al. (1994). Transplantation 57:974-978.

Leventhal J R, et al. (1995). Transplantation 59:294-300.

Lin S S, et al. (1998). J. Clin. Invest. 101:1745-1756.

Lin Y, et al. (1997). J. Immunol. 158:5658-5667.

Linsley P S, et al. (1991). J. Exp. Med. 173:721-730.

Liszewski M K and Atkinson J P (1998). In The Human Complement System in Health and Disease (Volanakis J E and Frank M M, eds.) pp. 149-165, Marcel Dekker, Inc. New York.

Liszewski M K, et al. (1996). Adv. Immunol. 61:201-283.

Lu C Y, et al. (1994). FASEB J. 8:1122-1130.

Luque I, et al. (2000). Hum. Immunol. 61:721-728.

Maher S E, et al. (1996). J. Immunol. 157:3838-3844.

Markowitz D, et al. (1988). J. Virol. 62:1120-1124.

Matsumiya G, et al. (1996). Xenotransplantation 3:76.

Matsumoto I and Osawa T (1969). Biochim. Biophys. Acta 194:180-189.

McCurry K R, et al. (1995). Nat. Med. 1:423-427.

McMahon A P and Bradley A (1990). Cell 62:1073-1085.

Millan M T, et al. (1997). Transplantation 63:421-429.

Miller A D and Buttimore C (1986). Mol. Cell. Biol. 6:2895-2902.

Miller et al. (1989). Biotechniques 7:981.

Montel A H, et al. (1995). Cell. Immunol. 160:104-114.

Morgan B P and Meri S (1994). Springer Semin. Immunopathol. 15:369-396.

Morgenstern J P and Land H (1990). Nucl. Acids Res. 18:3587-3596.

Mullis K, et al. (1986). Cold Spring Harb. Symp. Quant. Biol. 51:263-273.

Murray A G, et al. (1994). Immunity 1:57-63.

Ng S Y, et al. (1989). Nucl. Acids Res. 17:601-615.

Nicholson-Weller A, et al. (1982). J. Immunol. 129:184-189.

Pangburn M K and Müller-Eberhard H J (1983). Biochemistry 22:178-185.

Pearson T C, et al. (1994). Transplantation 57:1701-1706.

Pierson R N, et al. (1989). J. Exp. Med. 170:991-996.

Platt J L and Bach F H (1991). Curr. Opin. Immunol. 3:735-739.

Platt J L, et al. (1998). Xenotransplantation 5:169-175.

Pruitt S K, et al. (1994). Transplantation 57:363-370.

Robertson, in Robertson (ed.) (1987). Teratocarcinomas and Embryonic Stem Cells a Practical Approach, IRL Press, Eynsham, Oxford, England.

Robertson E, et al. (1986). Nature 323:445-448.

Rollins S A and Sims P J (1990). J. Immunol. 144:3478-3483.

Rollins S A, et al. (1991). J. Immunol. 146:2345-2351.

Rother R P, et al. (1994). J. Virol. 68:730-737.

Sambrook J, et al. (1989). Molecular Cloning: A laboratory manual, 2^{nd} edition. Cold Spring Harbor Press.

Sandrin M S, et al. (1995). Nat. Med. 1:1261-1267.

Seya T, et al. (1986). J. Exp. Med. 163:837-855.

Shapiro V S, et al. (1998). J. Immunol. 161:6455-6458.

Sheikh S, et al. (2000). Transplantation 70:917-928.

Simeonovic C J, et al. (1990). Transplantation 49:849-856.

Somerville C A and D'Apice A J F (1993). Kidney International Suppl. 42:S112-S121.

Steurer W, et al. (1995). J. Immunol. 155:1165-1174.

Sun H, et al. (1997). Transplantation 64:1838-1843.

Thompson C B, et al. (1989). Proc. Natl. Acad. Sci. USA 86:1333-1337.

Tran H M, et al. (1997). J. Immunol. 159:2232-2239.

Walunas T L, et al. (1994). Immunity 1:405-413.

Watier H, et al. (1996). Transpl. Immunol. 4:293-299.

Watzl C, et al. (2000). J. Immunol. 165:3545-3548.

Wecker H, et al. (1994). Xenotransplantation 1:8.

Wilson J L, et al. (1999). J. Immunol. 163:4207-4212.

Xia G, et al. (2000). Transplantation 70:86-93.

Yoneda N, et al. (1992). Leukemia 6:136-141.

Zhao J, et al. (1991). J. Biol. Chem. 266:13418-13422.

Zhong R, et al. (1996). Transplant. Proc. 28:762.

U.S. Pat. No. 4,405,712.

U.S. Pat. No. 4,650,764.

U.S. Pat. No. 4,861,719.

U.S. Pat. No. 4,873,191.

U.S. Pat. No. 4,980,289.

U.S. Pat. No. 5,032,407.

U.S. Pat. No. 5,112,767.

U.S. Pat. No. 5,124,263.

U.S. Pat. No. 5,135,916.

U.S. Pat. No. 5,166,065.

U.S. Pat. No. 5,434,131.

U.S. Pat. No. 5,624,837.

U.S. Pat. No. 5,627,264.

U.S. Pat. No. 5,773,253.

U.S. Pat. No. 5,844,095.

U.S. Pat. No. 5,847,082.

U.S. Pat. No. 5,851,795.

U.S. Pat. No. 5,869,050.

PCT Publication No. WO 85/05629.

PCT Publication No. WO 89/07150.

PCT Publication No. WO 90/02797.

PCT Publication No. WO 90/02806.

PCT Publication No. WO 90/08832.

PCT Publication No. WO 90/13641.

PCT Publication No. WO 92/05266.

PCT Publication No. WO 92/07943.

PCT Publication No. WO 92/11757.

PCT Publication No. WO 92/14829.

PCT Publication No. WO 93/02188.

PCT Publication No. WO 93/14188.

PCT Publication No. WO 99/07829

What is claimed is:

1. A chimeric protein comprising a first domain capable of inhibiting a cellular immune response and a second domain capable of inhibiting a humoral immune response.

2. The chimeric protein of claim 1 wherein said cellular immune response is a T-cell immune response or an NK cell immune response.

3. The chimeric protein of claim 1 wherein said humoral immune response is a complement mediated response.

4. The chimeric protein of claim 1 further comprising a cellular anchor moiety.

5. The chimeric protein of claim 4 wherein said moiety is a glycosyl-phosphatidylinositol (GPI) anchor.

6. The chimeric protein of claim 5 wherein said GPI anchor is bound to an amino acid residue on said second domain.

7. The chimeric protein of claim 1 wherein said second domain comprises C5b-9 inhibitory activity and wherein said first domain comprises i) T-cell inhibitory activity or ii) NK cell inhibitory activity.

8. The chimeric protein of claim 7, wherein the domain comprising C5b-9 inhibitory activity is i) a mammalian CD59 or ii) a mammalian CD59 comprising one or more conservative amino acid substitutions.

9. The chimeric protein of claim 8 wherein said mammalian CD59 is a human CD59.

10. The chimeric protein of claim 7, wherein the protein exhibits at least about 25% of the C5b-9 inhibitory activity of a naturally occurring wild-type C5b-9 inhibitor protein.

11. The chimeric protein of claim 1 wherein said second domain comprises a C3 inhibitory activity and wherein said first domain comprises i) T-cell inhibitory activity or ii) NK cell inhibitory activity.

12. The chimeric protein of claim 11 wherein the domain comprising C3 inhibitory activity is i) mammalian decay accelerating factor (DAF), CR1, MCP, or factor H, or ii) mammalian DAF comprising one or more conservative amino acid substitutions, CR1 comprising one or more conservative amino acid substitutions, MCP comprising one or more conservative amino acid substitutions, or factor H comprising one or more conservative amino acid substitutions.

13. The chimeric protein of claim 7, wherein the protein has at least about 25% of i) the T-cell inhibitory activity of a naturally occurring wild-type T-cell inhibitor protein or ii) the NK cell inhibitory activity of a naturally occurring wild-type NK cell inhibitor protein.

14. The chimeric protein of claim 7, wherein the domain having T-cell inhibitory activity or NK cell inhibitory activity is i) a mammalian CTLA4 or ii) a mammalian CTLA4 comprising one or more conservative amino acid substitutions.

15. The chimeric protein of claim 14, wherein the mammalian CTLA4 is selected from the group consisting of human CTLA4 and porcine CTLA4.

16. The chimeric protein of claim 1, wherein the protein comprises a linker region between said first domain and said second domain.

17. The chimeric protein of claim 16 wherein said linker region consists of Gly-Gly-Gly-Gly-Gly-Gly (SEQ ID NO:12) or Ala-Ser-Ala-Ser-Ala-Ser (SEQ ID NO:13).

18. The chimeric protein of claim 1 further comprising a leader sequence capable of transporting said chimeric protein outside of a cell.

19. The chimeric protein of claim 18 wherein said leader sequence is porcine or human.

20. The chimeric protein of claim 18 wherein said leader sequence is a CD59 leader sequence.

21. The chimeric protein of claim 18 wherein said leader sequence is a CTLA4 leader sequence.

22. The chimeric protein of claim 1 wherein said first domain is nearer to the amino terminus of said chimeric protein's sequence than is said second domain.

23. A chimeric DNA construct comprising i) a DNA sequence encoding a domain capable of inhibiting a cellular immune response and ii) a DNA sequence encoding a domain capable of inhibiting a humoral immune response.

24. The chimeric DNA construct of claim 23 wherein said cellular immune response is a T-cell response or an NK cell response.

25. The chimeric DNA construct of claim 23 wherein said humoral immune response is a complement mediated response.

26. The chimeric DNA construct of claim 23 wherein i) said domain capable of inhibiting a cellular immune response comprises T-cell inhibitory activity or NK cell inhibitory activity and ii) said domain capable of inhibiting a humoral immune response comprises C5b-9 inhibitory activity.

27. The chimeric DNA construct of claim 26, wherein the domain comprising C5b-9 inhibitory activity is i) a mammalian CD59 or ii) a mammalian CD59 comprising one or more conservative amino acid substitutions.

28. The chimeric DNA construct of claim 23 wherein said domain capable of inhibiting a cellular immune response comprises T-cell inhibitory activity or NK cell inhibitory activity and ii) said domain capable of inhibiting a humoral response comprises C3 inhibitory activity.

29. The chimeric DNA construct of claim 28 wherein said C3 inhibitory activity is i) mammalian DAF, CR1, MCP, or factor H, or ii) mammalian DAF comprising one or more conservative amino acid substitutions, CR1 comprising one or more conservative amino acid substitutions, MCP comprising one or more conservative amino acid substitutions, or factor H comprising one or more conservative amino acid substitutions.

30. The chimeric DNA construct of claim 26, wherein the domain comprising T-cell inhibitory activity or NK cell inhibitory activity is i) a mammalian CTLA4 or ii) a mammalian CTLA4 comprising one or more conservative amino acid substitutions.

31. The chimeric DNA construct of claim 30, wherein the mammalian CTLA4 is selected from the group consisting of human and porcine CTLA4.

32. A cloning vector comprising a DNA construct of claim 23.

33. The cloning vector of claim 32, wherein the cloning vector is a retroviral vector.

34. A host cell transformed by the vector of claim 32.

35. The host cell of claim 34 further comprising a transgene capable of expressing H-transferase.

36. A transgenic cell, tissue, organ or mammal comprising the chimeric protein of claim 1.

37. The transgenic cell, tissue or organ of claim 36 wherein said cell, tissue or organ is mammalian.

38. The transgenic cell, tissue, organ or mammal of claim 36 wherein said cell, tissue, organ or mammal is porcine.

39. The transgenic cell, tissue, organ or mammal of claim 36 further comprising a transgene capable of expressing H-transferase.

40. A method of producing a mammal, a mammalian organ, a mammalian tissue or mammalian cells, wherein said mammal is useful as an organ donor for a human or said organ, tissue or cells are useful for transplant into a human, wherein said method comprises inserting a nucleic acid into said mammal, organ, tissue or cells wherein said nucleic acid encodes a chimeric protein comprising a first domain capable of inhibiting a cellular immune response and a second domain capable of inhibiting a humoral immune response, wherein said protein is expressed in said mammal, organ, tissue or cells.

41. The method of claim 40 wherein said mammal, organ, tissue or cells are porcine.

42. The method of claim 40 wherein said first domain comprises a mammalian CTLA4.

43. The method of claim 40 wherein said second domain comprises a mammalian CD59, DAF, CR1, MCP or factor H.

44. The method of claim 40 wherein said mammal, mammalian organ, mammalian tissue or mammalian cells comprise a transgene capable of expressing H-transferase.

45. A nucleic acid comprising bases 112-486 of SEQ ID NO:5.

46. The nucleic acid of claim 45 comprising bases 112-669 of SEQ ID NO:5.

47. The nucleic acid of claim 45 comprising bases 1-486 of SEQ ID NO:5.

48. The nucleic acid of claim 45 comprising bases 1-669 of SEQ ID NO:5.

49. A nucleic acid encoding a polypeptide comprising amino acids 1-125 of SEQ ID NO:6.

50. The nucleic acid of claim 49 encoding a polypeptide comprising amino acids 1-186 of SEQ ID NO:6.

51. The nucleic acid of claim 49 encoding a polypeptide comprising amino acids −37 through 125 of SEQ ID NO:6.

52. The nucleic acid of claim 49 encoding a polypeptide comprising amino acids −37 through 186 of SEQ ID NO:6.

53. A polypeptide comprising amino acids 1-125 of SEQ ID NO:6.

54. The polypeptide of claim 53 comprising amino acids 1-186 of SEQ ID NO:6.

55. The polypeptide of claim 53 comprising amino acids −37 through 125 of SEQ ID NO:6.

56. The polypeptide of claim 53 comprising amino acids −37 through 186 of SEQ ID NO:6.


SEQUENCE LISTING (27 sequences)

SEQ ID NO: 1 - DNA, 833 bp, Artificial Sequence
  DNA used in the cloning of porcine CTLA4 - human CD59 chimeric mo lecules.
ggatcctgggcgccgccaggttctgtggacaatcacaatgggaatccaaggagggtctgtcctgttcgggctgctgctcgtcctagctgtcttctgccattcaggtcatagcctgcatatgcacgtggcccagcctgcagtggtgctggccaacagccggggtgttgccagctttgtgtgtgagtatgggtctgcaggcaaagctgccgaggtccgggtgacagtgctgcggcgggccggcagccagatgactgaagtctgtgccgcgacatatactgtggaggatgagttgaccttccttgatgactctacatgcactggcacctccaccgaaaacaaagtgaacctcaccatccaagggctgagagccgtggacactgggctctacatctgcaaggtggagctcctgtacccaccaccctactatgtgggtatgggcaacgggacccagatttatgtcattgatccagaaccatgcccagattctgatttcggtggcggtggcggtggcatgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcggaattc

SEQ ID NO: 2 - protein, 261 aa, Artificial Sequence
  DNA used in the cloning of porcine CTLA4 - human CD59 chimeric molecules.
MGIQGGSVLFGLLLVLAVFCHSGHSLHMHVAQPAVVLANSRGVASFVCEYGSAGKAAEVRVTVLRRAGSQMTEVCAATYTVEDELTFLDDSTCTGTSTENKVNLTIQGLRAVDTGLYICKVELLYPPPYYVGMGNGTQIYVIDPEPCPDSDFGGGGGGMQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 3 - DNA, 830 bp, Artificial Sequence
  DNA used in the cloning of human CTLA4 - human CD59 chimeric molecules.
ggatcctgggcgccgcaggttctgtggacaatcacaatgggaatccaaggagggtctgtcctgttcgggctgctgctcgtcctggctgtcttctgccattcaggtcatagcctgcagatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgacggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacgcctccgcctccgcctccctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcggaattcc

SEQ ID NO: 4 - protein, 260 aa, Artificial Sequence
  DNA used in the cloning of human CTLA4 - human CD59 chimeric molecules.
MGIQGGSVLFGLLLVLAVFCHSGHSLQMHVAQPAVVLASSRGIASFVCEYASPGKATEVRVTVLRQADSQVTEVCAATYMTGNELTFLDDSICTGTSSGNQVNLTIQGLRAMDTGLYICKVELMYPPPYYLGIGNGTQIYVIDPEPCPDSDASASASLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 5 - DNA, 672 bp, Porcine
  CDS (1)..(669)
atggctcgtttcggattccggagccatggggcttggctggagcttacttctaggacctggccctgtacagctctgttttctcttctcttcatccctgtcttctccaaagggatgcacgtggcccagcctgcagtggtgctggccaacagccggggtgttgccagctttgtgtgtgagtatgggtctgcaggcaaagctgccgaggtccgggtgacagtgctgcggcgggccggcagccagatgactgaagtctgtgccgcgacatatactgtggaggatgagttgaccttccttgatgactctacatgcactggcacctccaccgaaaacaaagtgaacctcaccatccaagggctgagagccgtggacactgggctctacatctgcaaggtggagctcctgtacccaccaccctactatgtgggtatgggcaacgggacccagatttatgtcattgatccagaaccatgcccagattctgatttcctgctctggatcctggcaacagttagttcagggttgtttttttacagcttcctcatcacagctgtttctttgagcaaaatgctaaagaaaagaagtcctcttactacaggggtctatgtgaaaaatgccccgacagagccagaatgtgaaaagcaatttcagccttattttattcccatcaactga

SEQ ID NO: 6 - protein, 223 aa, Porcine
MARFGFRSHGAWLELTSRTWPCTALFSLLFIPVFSKGMHVAQPAVVLANSRGVASFVCEYGSAGKAAEVRVTVLRRAGSQMTEVCAATYTVEDELTFLDDSTCTGTSTENKVNLTIQGLRAVDTGLYICKVELLYPPPYYVGMGNGTQIYVIDPEPCPDSDFLLWILATVSSGLFFYSFLITAVSLSKMLKKRSPLTTGVYVKNAPTEPECEKQFQPYFIPIN

SEQ ID NO: 7 - DNA, 672 bp, Homo sapiens
  CDS (1)..(669)
atggcttgccttggatttcagcggcacaaggctcagctgaacctggctgccaggacctggccctgcactctcctgttttttcttctcttcatccctgtcttctgcaaagcaatgcacgtggcccagcctgctgtggtactggccagcagccgaggcatcgccagctttgtgtgtgagtatgcatctccaggcaaagccactgaggtccgggtgacagtgcttcggcaggctgacagccaggtgactgaagtctgtgcggcaacctacatgacggggaatgagttgaccttcctagatgattccatctgcacgggcacctccagtggaaatcaagtgaacctcactatccaaggactgagggccatggacacgggactctacatctgcaaggtggagctcatgtacccaccgccatactacctgggcataggcaacggaacccagatttatgtaattgatccagaaccgtgcccagattctgacttcctcctctggatccttgcagcagttagttcggggttgtttttttatagctttctcctcacagctgtttctttgagcaaaatgctaaagaaaagaagccctcttacaacaggggtctatgtgaaaatgcccccaacagagccagaatgtgaaaagcaatttcagccttattttattcccatcaattga

SEQ ID NO: 8 - protein, 223 aa, Homo sapiens
MACLGFQRHKAQLNLAARTWPCTLLFFLLFIPVFCKAMHVAQPAVVLASSRGIASFVCEYASPGKATEVRVTVLRQADSQVTEVCAATYMTGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 9 - DNA, 476 bp, Homo sapiens
  CDS (80)..(463)
gaattccgggggctgagcgcagaagcggctcgaggctggaagaggatcctgggcgccgcaggttctgtggacaatcacaatgggaatccaaggagggtctgtcctgttcgggctgctgctcgtcctggctgtcttctgccattcaggtcatagcctgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcggaattc

SEQ ID NO: 10 - protein, 128 aa, Homo sapiens
MGIQGGSVLFGLLLVLAVFCHSGHSLQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 11 - protein, 314 aa, Homo sapiens
  mat_peptide (35)..()
MTVARPSVPAALPLLGELPRLLLLVLLCLPAVWGDCGLPPDVPNAQPALEGRTSFPEDTVITYKCEESFVKIPGEKDSVICLKGSQWSDIEEFCNRSCEVPTRLNSASLKQPYITQNYFPVGTVVEYECRPGYRREPSLSPKLTCLQNLKWSTAVEFCKKKSCPNPGEIRNGQIDVPGGILFGATISFSCNTGYKLFGSTSSFCLISGSSVQWSDPLPECREIYCPAPPQIDNGIIQGERDHYGYRQSVTYACNKGFTMIGEHSIYCTVNNDEGEWSGPPPECRGKSLTSKVPPTVQKPTTVNVPTTEVSPTSQ

SEQ ID NO: 12 - protein, 6 aa, Artificial Sequence
  Linker sequence.
GGGGGG

SEQ ID NO: 13 - protein, 6 aa, Artificial Sequence
  Linker sequence.
ASASAS

SEQ ID NO: 14 - DNA, 20 bp, Artificial Sequence
  5′ forward primer.
gcctgcagatgcacgtggcc

SEQ ID NO: 15 - DNA, 38 bp, Artificial Sequence
  3′ reverse primer.
ggctgcagggaggcggaggcggaggcgtcagaatctgg

SEQ ID NO: 16 - DNA, 25 bp, Artificial Sequence
  5′ forward primer.
cccmymmagccatggctygyyyygg

SEQ ID NO: 17 - DNA, 29 bp, Artificial Sequence
  3′ reverse primer.
cctcarttratrggaataaaataaggctg

SEQ ID NO: 18 - DNA, 30 bp, Artificial Sequence
  5′ forward primer.
ccatgcatatgcacgtggcccagcctgcag

SEQ ID NO: 19 - DNA, 59 bp, Artificial Sequence
  3′ reverse primer.
catgcatgccaccgccaccgccaccgaaatcagaatctgggcatggttctggatcaatg

SEQ ID NO: 20 - DNA, 33 bp, Artificial Sequence
  5′ forward primer.
gggctagcagccatggctcgtttcggattccgg

SEQ ID NO: 21 - DNA, 43 bp, Artificial Sequence
  3′ reverse primer.
cggatccgaaatcagaatctgggcatggttctggatcaatgac

SEQ ID NO: 22 - DNA, 28 bp, Artificial Sequence
  5′ forward primer.
ccatgcatatgcacgtggcccagcctgc

SEQ ID NO: 23 - DNA, 50 bp, Artificial Sequence
  3′ reverse primer.
ccatgcatggaggcggaggcggaggcatcagaatctgggcatggttctgg

SEQ ID NO: 24 - DNA, 846 bp, Artificial Sequence
  Chimeric porcine CTLA4-human CD59 with porcine CTLA4 leader sequence and (AS)3 linker region.
gaattcgcccttcccctccagccatggctcgtttcggattccggagccatggggcttggctggagcttacttctaggacctggccctgtacagctctgttttctcttctcttcatccctgtcttctccaaagggatgcacgtggcccagcctgcagtggtgctggccaacagccggggtgttgccagctttgtgtgtgagtatgggtctgcaggcaaagctgccgaggtccgggtgacagtgctgcggcgggccggcagccagatgactgaagtctgtgccgcgacatatactgtggaggatgagttgaccttccttgatgactctacatgcactggcacctccaccgaaaacaaagtgaacctcaccatccaagggctgagagccgtggacactgggctctacatctgcaaggtggagctcctgtacccaccaccctactatgtgggtatgggcaacgggacccagatttatgtcattgatccagaaccatgcccagattctgatgcctccgcctccgcctccatgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcggaattc

SEQ ID NO: 25 - protein, 270 aa, Artificial Sequence
  Chimeric porcine CTLA4-human CD59 with porcine CTLA4 leader sequence and (AS)3 linker region.
MARFGFRSHGAWLELTSRTWPCTALFSLLFIPVFSKGMHVAQPAVVLANSRGVASFVCEYGSAGKAAEVRVTVLRRAGSQMTEVCAATYTVEDELTFLDDSTCTGTSTENKVNLTIQGLRAVDTGLYICKVELLYPPPYYVGMGNGTQIYVIDPEPCPDSDASASASMQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP

SEQ ID NO: 26 - DNA, 849 bp, Artificial Sequence
  Chimeric porcine CTLA4-human CD59 with porcine CTLA4 leader sequence and (G)6 linker region.
gaattcgcccttcccctccagccatggctcgtttcggattccggagccatggggcttggctggagcttacttctaggacctggccctgtacagctctgttttctcttctcttcatccctgtcttctccaaagggatgcacgtggcccagcctgcagtggtgctggccaacagccggggtgttgccagctttgtgtgtgagtatgggtctgcaggcaaagctgccgaggtccgggtgacagtgctgcggcgggccggcagccagatgactgaagtctgtgccgcgacatatactgtggaggatgagttgaccttccttgatgactctacatgcactggcacctccaccgaaaacaaagtgaacctcaccatccaagggctgagagccgtggacactgggctctacatctgcaaggtggagctcctgtacccaccaccctactatgtgggtatgggcaacgggacccagatttatgtcattgatccagaaccatgcccagattctgatttcggtggcggtggcggtggcatgcagtgctacaactgtcctaacccaactgctgactgcaaaacagccgtcaattgttcatctgattttgatgcgtgtctcattaccaaagctgggttacaagtgtataacaagtgttggaagtttgagcattgcaatttcaacgacgtcacaacccgcttgagggaaaatgagctaacgtactactgctgcaagaaggacctgtgtaactttaacgaacagcttgaaaatggtgggacatccttatcagagaaaacagttcttctgctggtgactccatttctggcagcagcctggagccttcatccctaagtcggaattc

SEQ ID NO: 27 - protein, 271 aa, Artificial Sequence
  Chimeric porcine CTLA4-human CD59 with porcine CTLA4 leader sequence and (G)6 linker region.
MARFGFRSHGAWLELTSRTWPCTALFSLLFIPVFSKGMHVAQPAVVLANSRGVASFVCEYGSAGKAAEVRVTVLRRAGSQMTEVCAATYTVEDELTFLDDSTCTGTSTENKVNLTIQGLRAVDTGLYICKVELLYPPPYYVGMGNGTQIYVIDPEPCPDSDFGGGGGGMQCYNCPNPTADCKTAVNCSSDFDACLITKAGLQVYNKCWKFEHCNFNDVTTRLRENELTYYCCKKDLCNFNEQLENGGTSLSEKTVLLLVTPFLAAAWSLHP